(19) United States
(12) Patent Application Publication
(10) Pub. No.: US 2025/0195680 A1
(43) Pub. Date: Jun. 19, 2025

(54) CANCER TREATMENT WITH ANTI-CLAUDIN 18.2 ADC

(71) Applicants: Keymed Biosciences (Chengdu) Co., Ltd, Chengdu (CN); Shanghai Miracogen Inc., Shanghai (CN)

(72) Inventors: Bo CHEN, Chengdu (CN); Wei LIU, Chengdu (CN); Chaohong HU, Shanghai (CN)

(21) Appl. No.: 18/927,411

(22) Filed: Oct. 25, 2024

(30) Foreign Application Priority Data
Oct. 27, 2023 (WO) PCT/CN2023/127269

(51) Int. Cl. A61K 47/68

(52) U.S. Cl. CPC A61K 47/6851; A61K 47/68031; A61K 47/6889

(57) ABSTRACT

Provided herein is a method of treating cancer in a human subject, comprising administering to the subject an antibody-drug conjugate (ADC) comprising an antibody or antigen-binding fragment thereof which specifically binds human claudin 18.2 and monomethyl auristatin E, wherein the subject is administered at least one dose of the ADC of about 0.3 to about 3.4 mg/kg.

### RELATED APPLICATIONS

The present application claims the benefit of International App. No. PCT/CN2023/127269, filed on Oct. 27, 2023. The content of this application is incorporated herein by reference in its entirety.

### SEQUENCE LISTING

This application is accompanied by an XML file as a computer-readable format containing the sequence listing entitled, “2943_3150001_SequenceListing_ST26.XML” created on Oct. 22, 2024, with a file size of 13,200 bytes, the content of which is hereby incorporated by reference in its entirety.

### FIELD

Provided herein are dosage regimens for treating cancer with an antibody-drug conjugate (ADC) comprising an anti-claudin 18.2 antibody conjugated to monomethyl auristatin E (MMAE).

### BACKGROUND

The Claudin (CLDN) proteins participate in the formation of tight junctions between epithelial cells. CLDN proteins are transmembrane proteins with 4 transmembrane helices, with cytoplasmic N- and C-termini and two extracellular loops (ECLs). Different members of the Claudin family are expressed in different tissues.

Claudin 18 has two isoforms produced as a result of alternative splicing: CLDN18.1 and CLDN18.2. In healthy individuals, CLDN18.1 is expressed primarily in the lungs, in alveolar epithelial cells, whereas CLDN18.2 is expressed primarily in the stomach, in gastric mucosal membrane epithelial cells.

However, CLDN18.2 has been found to be expressed in a variety of cancers, including about 70% of gastric cancers and 50% of pancreatic cancers. Moreover, in healthy individuals CLDN18.2 is buried in tight junctions, but during malignant transformation is believed to become more exposed and thus therapeutically accessible. As a result, CLDN18.2 has been identified as a target for treatment of these cancers, particularly gastric cancer.

Gastric cancer is one of the most common cancers worldwide, with over a million cases diagnosed in 2020. Incidence of gastric cancer is particularly high in East Asia. The standard initial treatment for advanced or recurrent gastric cancer is chemotherapy. Although developments in treatment have improved the prognosis of gastric cancer patients in recent years, the 5-year overall survival rate is still only about 20%.

Targeted therapy has brought new hope for the treatment of recurrent/advanced gastric cancer. The anti-HER2 antibody trastuzumab combined with chemotherapy can benefit patients with HER2-positive cancer, but only about 15% of gastric cancers overexpress HER2. PD-1/PD-L1 inhibitors can also benefit gastric cancer patients, particularly those with PD-L1-positive tumours. Nonetheless, new therapies remain needed, and CLDN18.2 is an attractive target.

The anti-CLDN18.2 monoclonal antibody zolbetuximab has been found to be effective in treating gastric cancer, improving survival rates when combined with either CAPOX (Shah et al., Nature Medicine 29:2133-2141, 2023) or mFOLFOX6 (shitara et al., The Lancet 401 (10389): 1655-1668, 2023) compared to the chemotherapy alone, validating the approach of targeting CLDN18.2 in gastric cancer treatment.

WO 2020/211792 discloses a number of anti-CLDN18.2 antibodies that can be used in cancer therapy, including CM311. Antibody-drug conjugates (ADCs) based on the same antibody are disclosed in WO 2022/078523, including an ADC comprising CM311 conjugated to the potent antineoplastic agent monomethyl auristatin E (MMAE), known as CMG901. A phase I, dose escalation/expansion study of CMG901 has now been completed.

### SUMMARY

Provided herein is a method of treating cancer in a human subject, comprising administering to the subject an antibody-drug conjugate (ADC) comprising:
(i) an antibody or antigen-binding fragment thereof which specifically binds human claudin 18.2 and comprises a heavy chain variable region comprising VHCDR1, VHCDR2 and VHCDR3 and a light chain variable region comprising VLCDR1, VLCDR2 and VLCDR3, wherein:
   VHCDR1 comprises the amino acid sequence set forth in SEQ ID NO: 1;
   VHCDR2 comprises the amino acid sequence set forth in SEQ ID NO: 2;
   VHCDR3 comprises the amino acid sequence set forth in SEQ ID NO: 3;
   VLCDR1 comprises the amino acid sequence set forth in SEQ ID NO: 4;
   VLCDR2 comprises the amino acid sequence set forth in SEQ ID NO: 5; and
   VLCDR3 comprises the amino acid sequence set forth in SEQ ID NO: 6; and
(ii) monomethyl auristatin E (MMAE);
wherein the subject is administered at least one dose of the ADC of about 0.3 to about 3.4 mg/kg.

Relatedly, provided herein is the use of an antibody-drug conjugate (ADC) to treat cancer in a human subject, wherein the ADC comprises:
(i) an antibody or antigen-binding fragment thereof which specifically binds human claudin 18.2 and comprises a heavy chain variable region comprising VHCDR1, VHCDR2 and VHCDR3 and a light chain variable region comprising VLCDR1, VLCDR2 and VLCDR3, wherein:
   VHCDR1 comprises the amino acid sequence set forth in SEQ ID NO: 1;
   VHCDR2 comprises the amino acid sequence set forth in SEQ ID NO: 2;
   VHCDR3 comprises the amino acid sequence set forth in SEQ ID NO: 3;
   VLCDR1 comprises the amino acid sequence set forth in SEQ ID NO: 4;
   VLCDR2 comprises the amino acid sequence set forth in SEQ ID NO: 5; and
   VLCDR3 comprises the amino acid sequence set forth in SEQ ID NO: 6; and
(ii) monomethyl auristatin E (MMAE);
and wherein the subject is administered at least one dose of the ADC of about 0.3 to about 3.4 mg/kg.

Also relatedly, provided herein is the use of an antibody-drug conjugate (ADC) in the manufacture of a medicament for treating cancer in a human subject, wherein the ADC comprises:
(i) an antibody or antigen-binding fragment thereof which specifically binds human claudin 18.2 and comprises a heavy chain variable region comprising VHCDR1, VHCDR2 and VHCDR3 and a light chain variable region comprising VLCDR1, VLCDR2 and VLCDR3, wherein:
   VHCDR1 comprises the amino acid sequence set forth in SEQ ID NO: 1;
   VHCDR2 comprises the amino acid sequence set forth in SEQ ID NO: 2;
   VHCDR3 comprises the amino acid sequence set forth in SEQ ID NO: 3;
   VLCDR1 comprises the amino acid sequence set forth in SEQ ID NO: 4;
   VLCDR2 comprises the amino acid sequence set forth in SEQ ID NO: 5; and
   VLCDR3 comprises the amino acid sequence set forth in SEQ ID NO: 6; and
(ii) monomethyl auristatin E (MMAE);
and wherein the treatment comprises administering at least one dose of the ADC of about 0.3 to about 3.4 mg/kg to the subject.

Also relatedly, provided herein is an antibody-drug conjugate (ADC) for use in treating cancer in a human subject, wherein the ADC comprises:
(i) an antibody or antigen-binding fragment thereof which specifically binds human claudin 18.2 and comprises a heavy chain variable region comprising VHCDR1, VHCDR2 and VHCDR3 and a light chain variable region comprising VLCDR1, VLCDR2 and VLCDR3, wherein:
   VHCDR1 comprises the amino acid sequence set forth in SEQ ID NO: 1;
   VHCDR2 comprises the amino acid sequence set forth in SEQ ID NO: 2;
   VHCDR3 comprises the amino acid sequence set forth in SEQ ID NO: 3;
   VLCDR1 comprises the amino acid sequence set forth in SEQ ID NO: 4;
   VLCDR2 comprises the amino acid sequence set forth in SEQ ID NO: 5; and
   VLCDR3 comprises the amino acid sequence set forth in SEQ ID NO: 6; and
(ii) monomethyl auristatin E (MMAE);
and wherein the treatment comprises administering at least one dose of the ADC of about 0.3 to about 3.4 mg/kg to the subject.

Also relatedly, provided herein is a pharmaceutical composition for use in treating cancer in a subject, wherein the composition comprises an antibody-drug conjugate (ADC) comprising:
(i) an antibody or antigen-binding fragment thereof which specifically binds human claudin 18.2 and comprises a heavy chain variable region comprising VHCDR1, VHCDR2 and VHCDR3 and a light chain variable region comprising VLCDR1, VLCDR2 and VLCDR3, wherein:
   VHCDR1 comprises the amino acid sequence set forth in SEQ ID NO: 1;
   VHCDR2 comprises the amino acid sequence set forth in SEQ ID NO: 2;
   VHCDR3 comprises the amino acid sequence set forth in SEQ ID NO: 3;
   VLCDR1 comprises the amino acid sequence set forth in SEQ ID NO: 4;
   VLCDR2 comprises the amino acid sequence set forth in SEQ ID NO: 5; and
   VLCDR3 comprises the amino acid sequence set forth in SEQ ID NO: 6; and
(ii) monomethyl auristatin E (MMAE);
and wherein the treatment comprises administering at least one dose of the ADC of about 0.3 to about 3.4 mg/kg to the subject.

### DETAILED DESCRIPTION

The present inventors have identified particularly effective dosage regimens for the treatment of cancer with an antibody-drug conjugate (ADC) targeting Claudin 18.2 (CLDN18.2).

The ADC used herein comprises an antibody, or an antigen-binding fragment of an antibody, conjugated (that is to say, attached to) the cytotoxin monomethyl auristatin E (MMAE).

### Antibodies

As defined herein, in line with standard terminology in the art, an antibody is an antigen-binding protein comprising two heavy chains and two light chains. The light chains are shorter (and thus lighter) than the heavy chains. The heavy chains comprise an N-terminal heavy chain variable domain (V_{H}), and the light chains comprise an N-terminal light chain variable domain (V_{L}). The heavy and light chains each comprise one or more constant domains C-terminal to the respective variable domain.

Both the light and heavy chains of an antibody comprise three hypervariable complementarity-determining regions (CDRs), as set out herebelow. In a pair of a light chain and a heavy chain, the CDRs of the two chains form the antigen-binding site. The CDR sequences determine the specificity of an antibody. The three CDRs of a heavy chain are known as VHCDR1, VHCDR2 and VHCDR3, from N-terminus to C-terminus, and the three CDRs of a light chain are known as VLCDR1, VLCDR2 and VLCDR3, from N-terminus to C-terminus. Framework regions are located in between the CDRs and between the CDRs and ends of the variable domains. Antigen-binding fragments of antibodies are fragments or synthetic constructs comprising one or more antigen-binding sites of an antibody, but not the entire antibody. Generally an antigen-binding fragment of an antibody comprises the entire V_{L} and V_{H} domain sequences, but lacks at least part of the heavy and/or light chain constant domains.

The antibody, or antigen-binding fragment thereof (henceforth simply “fragment thereof”, or “antibody fragment”), used in the ADC specifically binds human CLDN18.2. Human CLDN18.2 has the UniProt accession number P56856-2, and the amino acid sequence set forth in SEQ ID NO: 11. Thus the antibody or fragment thereof specifically binds a protein with the sequence of SEQ ID NO: 11.

An antibody which binds specifically to human CLDN18.2 is an antibody which binds to human CLDN18.2 with a greater affinity than that with which it binds to other molecules, or at least most other molecules. In particular, an antibody which binds specifically to human CLDN18.2 either does not bind human CLDN18.1 (UniProt P56856-1, SEQ ID NO: 12), or binds human CLDN18.1 with much lower affinity than human CLDN18.2, e.g. it may bind human CLDN18.2 with an affinity which is one or more orders of magnitude higher than the affinity with which it binds human CLDN18.1.

An antibody which specifically binds human CLDN18.2 may display cross-reactivity with CLDN18.2 from other species. Regardless, the skilled person can readily determine whether an antibody or fragment thereof specifically binds human CLDN18.2 using standard techniques in the art, e.g. ELISA, Western-blot, surface plasmon resonance (SPR), etc.

The antibody or fragment thereof used in the present ADC comprises a heavy chain variable region comprising VHCDR1, VHCDR2 and VHCDR3 and a light chain variable region comprising VLCDR1, VLCDR2 and VLCDR3, wherein:
VHCDR1 comprises or consists of the amino acid sequence set forth in SEQ ID NO: 1 (GGSISSNYAWN);
VHCDR2 comprises or consists of the amino acid sequence set forth in SEQ ID NO: 2 (YIYYSGNTNYNPSLKS);
VHCDR3 comprises or consists of the amino acid sequence set forth in SEQ ID NO: 3 (SYYGNSFIY);
VLCDR1 comprises or consists of the amino acid sequence set forth in SEQ ID NO: 4 (KSSQSLLNSGNQKNYLT);
VLCDR2 comprises or consists of the amino acid sequence set forth in SEQ ID NO: 5 (WASTRES); and
VLCDR3 comprises or consists of the amino acid sequence set forth in SEQ ID NO: 6 (QNAYSFPWT).

The antibody or fragment thereof used in the ADC for use herein is capable of mediating endocytosis of CLDN18.2 expressed on the surface of a target cell. That is to say, upon binding of the antibody or fragment thereof to CLDN18.2 on the surface of a cell, the CLDN18.2 and bound antibody are internalised into the cell by endocytosis. Whether an antibody induces endocytosis upon binding to a cell surface protein can be determined by e.g. confocal microscopy using a fluorescent-tagged antibody.

As set out above, the present ADC comprises an antibody or fragment thereof. An “antibody” as referred to herein is an immunoglobulin having the features described hereinbefore. Antigen-binding fragments of antibodies are discussed in Rodrigo et al., Antibodies, Vol. 4 (3), p. 259-277, 2015. Antibody fragments which may be used herein include, for example, Fab, F(ab′)2, Fab′ and Fv fragments. Fab fragments are discussed in Nelson, mAbs 2 (1): 77-83, 2010. A Fab fragment consists of the antigen-binding domain of an antibody, i.e. an individual antibody may be seen to contain two Fab fragments, each consisting of a light chain and its conjoined N-terminal section of the heavy chain. Thus a Fab fragment contains an entire light chain and the V_{H} and C_{H}1 domains of the heavy chain to which it is bound. Fab fragments may be obtained by digesting an antibody with papain.

F(ab′)2 fragments consist of the two Fab fragments of an antibody, plus the hinge regions of the heavy domains, including the disulphide bonds linking the two heavy chains together. In other words, a F(ab′)2 fragment can be seen as two covalently joined Fab fragments. F(ab′)2 fragments may be obtained by digesting an antibody with pepsin.

Reduction of F(ab′)2 fragments yields two Fab′ fragments, which can be seen as Fab fragments containing an additional sulfhydryl group which can be useful for conjugation of the fragment to other molecules.

Fv fragments consist of just the variable domains of the light and heavy chains. These are not covalently linked and are held together only weakly by non-covalent interactions. Fv fragments can be modified to produce a synthetic construct known as a single chain Fv (scFv) molecule. Such a modification is typically performed recombinantly, by engineering the antibody gene to produce a fusion protein in which a single polypeptide comprises both the V_{H} and V_{L} domains. scFv fragments generally include a peptide linker covalently joining the V_{H} and V_{L} regions, which contributes to the stability of the molecule. The linker may comprise from 1 to 20 amino acids, such as for example 1, 2, 3 or 4 amino acids, 5, 10 or 15 amino acids, or other intermediate numbers in the range 1 to 20 as convenient. The peptide linker may be formed from any generally convenient amino acid residues, such as glycine and/or serine. One example of a suitable linker is Gly₄Ser (Gly-Gly-Gly-Gly-Ser, SEQ ID NO: 13). Multimers of such linkers may be used, such as for example a dimer, a trimer, a tetramer or a pentamer, e.g. (Gly₄Ser)₂, (Gly₄Ser)₃, (Gly₄Ser)₄ or (Gly₄Ser)₅. However, it is not essential that a linker be present, and the V_{L} domain may be linked to the V_{H} domain by a peptide bond. An scFv is herein defined as an antibody fragment, or antigen-binding fragment of an antibody.

The antibody or antibody fragment used in the ADC may be humanised. A “humanised” antibody is an antibody derived from non-human germline immunoglobulin sequences, but which has been modified to replace non-human sequences with human ones. A humanised antibody may be derived, for instance, from mouse, rat, rabbit, etc., germline immunoglobulin sequences. Indeed, a humanised antibody may be derived from the germline immunoglobulin sequences of any non-human animal. As defined herein, an antibody is considered humanised if at least one of the V_{H} and V_{L} domains is humanised. In particular, a humanised antibody may comprise a humanised V_{H} sequence and a humanised V_{L} sequence.

In a humanised variable domain, a non-human variable domain sequence is modified to replace the non-human (e.g. murine) framework sequences with human framework sequences, such that, generally, the only non-human sequences in the antibody are the CDR sequences (though the CDR sequences may also be modified during the humanisation process). Antibody humanisation is generally performed by a process known as CDR grafting, though any other technique in the art may be used. CDR grafting is well described in Williams, D. G. et al., Antibody Engineering Vol. 1, edited by R. Kontermann and S. Dubel, Chapter 21, pp. 319-339, 2010. In this process, humanisation of non-human variable domains involves intercalating the non-human CDRs from each immunoglobulin chain within the framework regions of the most appropriate human variable region. This is done by aligning the non-human variable domains with databases of known human variable domains (e.g. IMGT or Kabat). Appropriate human framework regions are identified from the best aligned variable domains, e.g. domains with high sequence identity between the human and non-human framework regions, domains containing CDRs of the same length, domains having the most similar structures (based on homology modelling), etc. The non-human CDR sequences are then grafted into the lead human framework sequences at the appropriate locations using recombinant DNA technology, and the humanised antibodies then produced and tested for binding to the target antigen. The process of antibody humanisation is known and understood by the skilled individual, who can perform the technique without further instruction. Antibody humanisation services are also offered by a number of commercial companies, e.g. GenScript (USA/China) and LifeArc (UK). Humanised antibody fragments can be easily obtained from humanised antibodies, as described above.

In the present case, the CDR sequences of SEQ ID NOs: 1-6 were originally derived from a murine antibody (though are slightly modified compared to the murine parent antibody). An antibody comprising the CDR sequences of SEQ ID NOs: 1-6 grafted into human framework sequences is a humanised antibody. Thus the antibody used in the ADC may comprise a V_{L} and/or a V_{H} comprising human framework sequences.

In particular embodiments, the antibody or fragment thereof comprises:
(a) a heavy chain variable domain comprising or consisting of the amino acid sequence set forth in SEQ ID NO: 7, or a variant thereof having at least 80, 85, 90 or 95% identity to SEQ ID NO: 7; and
(b) a light chain variable domain comprising or consisting of the amino acid sequence set forth in SEQ ID NO: 8, or a variant thereof having at least 80, 85, 90 or 95% identity to SEQ ID NO: 8.

In particular, the antibody or fragment thereof may comprise a heavy chain variable domain comprising or consisting of the amino acid sequence set forth in SEQ ID NO: 7, and a light chain variable domain comprising or consisting of the amino acid sequence set forth in SEQ ID NO: 8.

When the antibody or antigen-binding fragment thereof comprises a V_{H} with at least 80% identity to SEQ ID NO: 7, but less than 100% identity to SEQ ID NO: 7, this is subject to the proviso that the CDRs are as defined above, i.e. that VHCDR1 comprises or consists of the amino acid sequence of SEQ ID NO: 1, VHCDR2 comprises or consists of the amino acid sequence of SEQ ID NO: 2 and VHCDR3 comprises or consists of the amino acid sequence of SEQ ID NO: 3. That is to say, when the heavy chain variable region comprises a variant of SEQ ID NO: 7, all variation in the heavy chain variable domain sequence relative to SEQ ID NO: 7 is found within the framework regions.

Similarly, when the antibody or antigen-binding fragment thereof comprises a V_{L} with at least 80% identity to SEQ ID NO: 8, but less than 100% identity to SEQ ID NO: 8, this is subject to the proviso that the CDRs are as defined above, i.e. that VLCDR1 comprises or consists of the amino acid sequence of SEQ ID NO: 4, VLCDR2 comprises or consists of the amino acid sequence of SEQ ID NO: 5 and VLCDR3 comprises or consists of the amino acid sequence of SEQ ID NO: 6. That is to say, when the light chain variable domain comprises a variant of SEQ ID NO: 8, all variation in the light chain variable domain sequence relative to SEQ ID NO: 8 is found within the framework regions.

In embodiments in which the ADC comprises an antibody, the heavy and light chains of the antibody each comprise a constant region. The constant regions are generally human constant regions.

As is well known in the art, antibodies may belong to a number of different isotypes, with the isotype of an antibody being determined by the sequence of its heavy chain constant region. In humans, the antibody isotypes are IgG, IgE, IgM, IgA and IgD. Some isotypes may be divided into further subtypes, e.g. there are four sub-types of IgG antibodies: IgG1, IgG2, IgG3 and IgG4. When an antibody is used in the methods herein, the antibody may be of any isotype, i.e. an IgG, IgE, IgM, IgA or IgD antibody may be used. In particular, the antibody may be an IgG antibody. When an IgG antibody is used it may be of any sub-type, i.e. IgG1, IgG2, IgG3 or IgG4 antibody may be used. In particular, the antibody may be an IgG1 antibody. In certain embodiments, the antibody is of the human IgG1 isotype (i.e. the antibody may comprise a human IgG1 constant domain).

In humans, the light chain constant region of an antibody may be a kappa or lambda (κ or λ) constant region, and thus a human antibody light chain may be a κ or λ light chain. An antibody used herein may comprise a κ or λ light chain. In particular, the antibody may comprise a κ light chain. In certain embodiments, the antibody comprises a light chain comprising a human κ constant domain.

The antibody for use herein may comprise:
(i) a heavy chain comprising or consisting of the amino acid sequence set forth in SEQ ID NO: 9, or a variant thereof having at least 80, 85, 90 or 95% sequence identity thereto; and
(ii) a light chain comprising or consisting of the amino acid sequence set forth in SEQ ID NO: 10, or a variant thereof having at least 80, 85, 90 or 95% sequence identity thereto.

As set out above, when the antibody comprises a heavy chain which is a variant of SEQ ID NO: 9, this is subject to the proviso that the CDRs are as defined above, i.e. that VHCDR1 comprises the amino acid sequence of SEQ ID NO: 1, VHCDR2 comprises the amino acid sequence of SEQ ID NO: 2 and VHCDR3 comprises the amino acid sequence of SEQ ID NO: 3. That is to say, when the heavy chain comprises a variant of SEQ ID NO: 9, all variation in the heavy chain sequence relative to SEQ ID NO: 9 is found within the constant domain and the framework regions of the variable domain.

Similarly, when the antibody comprises a light chain which is a variant of SEQ ID NO: 10, this is subject to the proviso that the CDRs are as defined above, i.e. that VLCDR1 comprises the amino acid sequence of SEQ ID NO: 4, VLCDR2 comprises the amino acid sequence of SEQ ID NO: 5 and VLCDR3 comprises the amino acid sequence of SEQ ID NO: 6. That is to say, when the light chain comprises a variant of SEQ ID NO: 10, all variation in the light chain sequence relative to SEQ ID NO: 10 is found within the constant domain and the framework regions of the variable domain.

The antibody with the heavy chain amino acid sequence set forth in SEQ ID NO: 9 and the light chain amino acid sequence SEQ ID NO: 10 is referred to herein as CM311.

Sequence identity of variants of the sequences set out above may be assessed by any convenient method. However, for determining the degree of sequence identity between sequences, computer programmes that make pairwise or multiple alignments of sequences are useful, for instance EMBOSS Needle or EMBOSS stretcher (both Rice, P. et al., Trends Genet. 16, (6) pp. 276-277, 2000) may be used for pairwise sequence alignments while Clustal Omega (Sievers F et al., Mol. Syst. Biol. 7:539, 2011) or MUSCLE (Edgar, R. C., Nucleic Acids Res. 32 (5): 1792-1797, 2004) may be used for multiple sequence alignments, though any other appropriate programme may be used. Whether the alignment is pairwise or multiple, it must be performed globally (i.e. across the entirety of the reference sequence) rather than locally.

Sequence alignments and % identity calculations may be determined using for instance standard Clustal Omega parameters: matrix Gonnet, gap opening penalty 6, gap extension penalty 1. Alternatively the standard EMBOSS Needle parameters may be used: matrix BLOSUM62, gap opening penalty 10, gap extension penalty 0.5. Any other suitable parameters may alternatively be used.

For the purposes of this application, where there is dispute between sequence identity values obtained by different methods, the value obtained by global pairwise alignment using EMBOSS Needle with default parameters shall be considered valid.

Variants of the sequences set out herein (i.e. sequences with at least 80% sequence identity to SEQ ID NO: 7, 8, 9 or 10) may be obtained by substitution, deletion or insertion of amino acid residues relative to the original sequences.

When a sequence is modified by substitution of a particular amino acid residue, the substitution may be a conservative amino acid substitution. The term “conservative amino acid substitution”, as used herein, refers to an amino acid substitution in which one amino acid residue is replaced with another amino acid residue having a similar side chain. Amino acids with similar side chains tend to have similar properties, and thus a conservative substitution of an amino acid important for the structure or function of a polypeptide may be expected to affect polypeptide structure/function less than a non-conservative amino acid substitution at the same position. Families of amino acid residues having similar side chains have been defined in the art, including basic side chains (e.g. lysine, arginine, histidine), acidic side chains (e.g. aspartic acid, glutamic acid), uncharged polar side chains (e.g. asparagine, glutamine, serine, threonine, tyrosine), non-polar side chains (e.g. glycine, cysteine, alanine, valine, leucine, isoleucine, proline, phenylalanine, methionine, tryptophan) and aromatic side chains (e.g. tyrosine, phenylalanine, tryptophan, histidine). Thus a conservative amino acid substitution may be considered to be a substitution in which a particular amino acid residue is substituted for a different amino acid in the same family. However, a substitution of an amino acid residue may equally be a non-conservative substitution, in which one amino acid is substituted for another with a side-chain belonging to a different family.

Where an antibody or fragment thereof is used which comprises a variant of the CM311 variable domain sequences (i.e. a variable domain which is a variant of SEQ ID NO: 7 or SEQ ID NO: 8, as set out above) or full chain sequences (i.e. a heavy or light chain which is a variant of SEQ ID NO: 9 or SEQ ID NO: 10), the variant may have equivalent activity to CM311 or a corresponding fragment thereof. For instance, an antibody which is a variant of CM311 (i.e. an antibody comprising a variant sequence as described above) may bind CLDN18.2 with an affinity which is equivalent to CM311, which is not lower than the affinity with which CM311 binds CLDN18.2, or which is not substantially lower than the affinity with which CM311 binds CLDN18.2. For instance, a variant of CM311 may be considered to bind CLDN18.2 with an affinity which is not substantially lower than the affinity with which CM311 binds CLDN18.2 if the variant of CM311 binds CLDN18.2 with an affinity which is reduced by no more than 5%, 10%, 15%, 20% or 25% compared to that of CM311.

The antibody or fragment thereof for use herein may be synthesised by any method known in the art. In particular, the antibody or fragment thereof may be synthesised using a protein expression system, such as a cellular expression system using prokaryotic (e.g. bacterial) host cells or eukaryotic (e.g. yeast, fungus, insect or mammalian) host cells. Cells which may be used in the production of the antibody or fragment thereof are discussed further below. An alternative protein expression system is a cell-free, in vitro expression system, in which a nucleotide sequence encoding the specific binding molecule is transcribed into mRNA, and the mRNA translated into a protein, in vitro. Cell-free expression system kits are widely available, and can be purchased from e.g. ThermoFisher Scientific (USA). Alternatively, antibodies and fragments thereof may be chemically synthesised in a non-biological system. Liquid-phase synthesis or solid-phase synthesis may be used to generate polypeptides which may form or be comprised within the antibody or fragment thereof used herein. The skilled person can readily produce antibodies or fragments thereof using appropriate methodology common in the art.

In particular, the antibody or fragment thereof may be recombinantly expressed in mammalian cells, such as CHO cells. Other suitable mammalian cells for production of the antibody or fragment thereof for use herein include monkey kidney cells (e.g. COS-7), HEK293 Hela cells, baby hamster kidney (BHK) cells, human hepatocellular carcinoma cells (e.g. Hep G2), and a number of other cell lines including the mouse myeloma cell lines NSO and SP2/0.

The host cell, when cultured under appropriate conditions, synthesises the antibody or antigen-binding fragment thereof for use herein that can subsequently be collected from the culture medium (if the host cell secretes it into the medium) or directly from the host cell producing it (if it is not secreted). Thus the antibody or fragment thereof may be isolated from synthesis, as discussed above. The host cell line which produces the antibody or fragment thereof for use herein may stably express the antibody or fragment thereof or transiently express the antibody or fragment thereof.

### Antibody-Drug Conjugates

The ADC for use herein comprises an antibody or fragment thereof, as described above, conjugated to MMAE. That is to say, the antibody or fragment thereof is covalently joined to MMAE. Throughout this section describing the ADC, the term “antibody” encompasses antibody fragments.

MMAE has the structure set out in formula I, below (in which the wavey line indicates the antibody to which MMAE is bound, optionally including a linker between the antibody and the MMAE).

### Formula I:

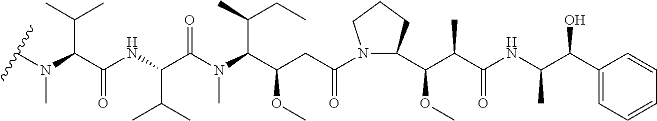 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

As mentioned above, MMAE may be joined to the antibody by a linker. A linker, as defined herein, is any chemical group or entity (which may be a peptide) which joins the MMAE to the antibody. The linker may join the MMAE to any functional group on the antibody, e.g. an amino group, carboxyl group, hydroxyl group or thiol group. The linker may join the MMAE to a side chain of the antibody fragment or to a terminus of an antibody chain. Generally, MMAE is joined to the antibody via thiol groups.

Linkers suitable for use in ADCs are known in the art, and any suitable linker may be used herein. In some embodiments, the linker is protease-cleavable. That is to say, the linker may be susceptible to cleavage by an intracellular protease, particularly an endosomal or lysosomal protease, such that upon endocytosis of the ADC by a target cell, the linker is cleaved and the MMAE released. In some embodiments, the linker is a cathepsin-cleavable linker, i.e. a linker susceptible to cleavage by a cathepsin protease, e.g. cathepsin B. Following release from the antibody, MMAE is transported into the cytoplasm where it binds to tubulin and inhibits its polymerization, thereby blocking mitosis, inhibiting tumour cell proliferation and leading to tumour cell death.

In some embodiments, the linker is selected from 6-maleimidohexanoyl (MC), maleimidopropionyl (MP), N-succinimidyl 4-(2-pyridylthio) valerate (SPP), 4-(N-maleimidomethyl)-cyclohexan-1-formyl (MCC), N-succinimidyl (4-iodo-acetyl) aminobenzoate (SIAB), and 6-maleimidocaproyl-valine-citrulline-p-aminobenzyloxycarbonyl (MC-vc-PAB). In particular embodiments, the linker is MC-vc-PAB. MC-vc-PAB has the structure set out in Formula II below:

### Formula II

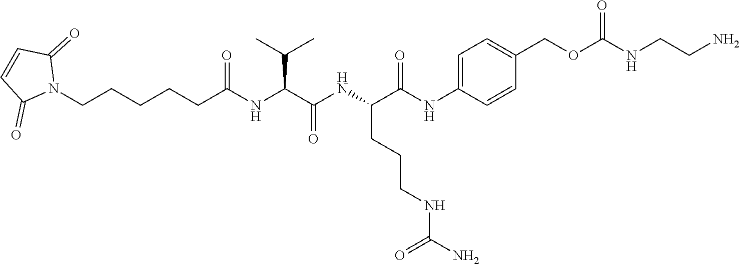 [SMILES: CC(C)[C@H](NC(=O)CCCCCN1C(=O)C=CC1=O)C(=O)N[C@@H](CCCNC(N)=O)C(=O)Nc1ccc(COC(=O)NCCN)cc1]

In some embodiments, the ADC comprises an antibody or fragment thereof comprising the heavy chain variable domain of SEQ ID NO: 7 and the light chain variable domain of SEQ ID NO: 8, conjugated to MMAE by a MC-vc-PAB linker.

In some embodiments, the ADC comprises an antibody comprising the heavy chain of SEQ ID NO: 9 and the light chain of SEQ ID NO: 10, conjugated to MMAE by a MC-vc-PAB linker.

The antibody in the ADC may be conjugated to e.g. 3, 4 or 5 MMAE molecules. The number of MMAE molecules attached to the antibody in the ADC is referred to as the drug-antibody ratio (DAR). It should be noted that the ADC used herein is generally provided in a pharmaceutical composition containing multiple ADC molecules, which may have the same or different DAR values. That is to say, in some embodiments, provided herein are pharmaceutical compositions comprising the ADC described above, in which the ADC molecules all comprise the same number of MMAE moieties. In other embodiments, provided herein are pharmaceutical compositions comprising the ADC described above, in which the ADC molecules comprise different numbers of MMAE moieties.

The average number of MMAE moieties in the ADC molecules in a composition thereof is referred to herein as the average DAR. This may be referred to as the average DAR of a composition comprising the ADC, or as the average DAR of the ADC (i.e. reference to the average DAR of the ADC is shorthand for reference to the average DAR of a composition comprising the ADC, i.e. a composition comprising a population of the ADC). The average DAR for a composition of the ADC can be determined by any conventional means in the art, e.g. mass spectrometry or HPLC. WO 2022/078523 describes determination of the average DAR of a composition of the ADC by hydrophobic interaction chromatography-HPLC (HIC-HPLC).

In some embodiments, the ADC has an average DAR of 3 to 4.5, e.g. 3 to 4.4, 3 to 4.3, 3 to 4.2, 3 to 4.1, 3 to 4, 3.1 to 4.5, 3.1 to 4.4, 3.1 to 4.3, 3.1 to 4.2, 3.1 to 4.1, 3.1 to 4, 3.2 to 4.5, 3.2 to 4.4, 3.2 to 4.3, 3.2 to 4.2, 3.2 to 4.1, 3.2 to 4, 3.3 to 4.5, 3.3 to 4.4, 3.3 to 4.3, 3.3 to 4.2, 3.3 to 4.1, 3.3 to 4, 3.4 to 4.5, 3.4 to 4.4, 3.4 to 4.3, 3.4 to 4.2, 3.4 to 4.1, 3.4 to 4, 3.5 to 4.5, 3.5 to 4.4, 3.5 to 4.3, 3.5 to 4.2, 3.5 to 4.1, 3.5 to 4, 3.6 to 4.5, 3.6 to 4.4, 3.6 to 4.3, 3.6 to 4.2, 3.6 to 4.1 or 3.6 to 4.

In particular embodiments, the ADC has an average DAR of 3.3 to 4.3, 3.4 to 4.2, 3.5 to 4.1, 3.6 to 4 or 3.7 to 3.9. In a particular embodiment, the ADC has an average DAR of about 3.8. An average DAR of about 3.8 may encompass an average DAR of e.g. 3.75 to 3.85, 3.76 to 3.84, 3.77 to 3.83, 3.78 to 3.82, 3.79 to 3.81. In some embodiments, the average DAR is 3.80.

As noted above, MMAE is generally attached to the antibody via thiol groups of its cysteine residues. In naturally occurring antibodies, many cysteine thiol groups are unavailable for conjugation to other moieties (such as drug molecules) as they exist in the context of disulphide bridges. In some cases, additional reactive thiol groups can be generated by treating the antibody with a reducing agent such as dithiothreitol (DTT) or tris (2-carboxyethyl) phosphine (TCEP), thereby increasing the obtainable DAR.

The ADC may be used in the context of a salt of the ADC described above, e.g. a salt of an inorganic acid such as a hydrochloride, hydrobromide, hydroiodide, nitrate, bicarbonate, carbonate, sulfate or phosphate salt; or a salt of an organic acid salt such as a formate, acetate, propionate, benzoate, maleate, fumarate, succinate, tartrate, citrate, ascorbate, α-ketoglutarate, α-glycerophosphate, alkyl sulfonate or aryl sulfonate salt. Examples of suitable alkyl sulfonate salts include methanesulfonate and ethanesulfonate. Examples of suitable aryl sulfonate salts are benzenesulfonate and p-toluenesulfonate.

The ADC, or salt thereof, may be used in the context of a solvate. The term “solvate” refers to solid or liquid forms of the ADC formed by coordination of the ADC with solvent molecules. A particularly suitable form of the ADC for use herein is a hydrate, which is a solvate with coordinated water molecules.

### Subjects

The subject (or patient) is a human suffering from cancer (i.e. who has been diagnosed with cancer). The cancer is generally a solid cancer. Generally the cancer (particularly solid cancer) expresses CLDN18.2 (by which is meant that at least some of the cells of the cancer express CLDN18.2). CLDN18.2 expression can be identified by any technique known in the art, e.g. immunohistochemistry (IHC) or qPCR.

In particular embodiments, the cancer is gastric cancer or gastroesophageal junction (GEJ) cancer. In other embodiments, the cancer is pancreatic cancer. The cancer may be any type of cancer, particularly an adenocarcinoma, e.g. gastric adenocarcinoma, GEJ adenocarcinoma or pancreatic ductal adenocarcinoma.

The cancer may be of any stage, but in particular may be an advanced cancer, e.g. a stage III or stage IV cancer. For example, the cancer may be a metastatic cancer or a locally unresectable cancer. In other embodiments, the cancer may be a borderline resectable cancer. Alternatively, the cancer may be an early-stage cancer, i.e. a non-metastatic and/or resectable cancer, e.g. a stage I or stage II cancer. Cancer stages referred to herein are those of the TNM staging system, which is well known in the art (see e.g. Rosen & Sapra, TNM Classification, in: StatPearls, Treasure Island (FL): StatPearls Publishing; 2023 January).

In some embodiments, the cancer does not express human epidermal growth factor receptor 2 (HER2). HER2 has the UniProt accession number P04626. HER2-positive cancers may be treated with drugs such as antibodies or ADCs which target HER2, e.g. trastuzumab or trastuzumab deruxtecan, and thus for HER2-positive cancers suitable alternative treatment options may exist, at least in the first instance. Nonetheless, HER2 expression is not believed to impact efficacy of the ADC provided herein, which is thus suitable for treating HER2-positive and HER2-negative cancers. Indeed, treatment with the ADC provided herein may be agnostic of HER2 expression.

HER2 expression by the cancer may be determined by any suitable technique, e.g. IHC or qPCR as mentioned above.

The subject may be at any stage of treatment. For example, the ADC used herein may be used as a first line drug (i.e., the subject may not have received any prior cancer therapeutic/line of treatment for their cancer), a second line drug (i.e. the subject may have received one prior cancer therapeutic/line of treatment for their cancer) or a third or further line drug (i.e. the subject may have received two or more prior cancer therapeutics/lines of treatment for their cancer).

### Treatment Regimens

As set out above, the subject treated with the ADC provided herein is administered at least one dose of the ADC of about 0.3 to about 3.4 mg/kg. Generally, the subject treated with the ADC is administered more than one dose of the ADC.

For subjects receiving more than one dose of the ADC, the frequency of the dosing may be determined by the subject's physician, but in general a dose of the ADC may be administered to the subject about every 2 weeks, every 3 weeks, every 4 weeks, every 5 weeks or every 6 weeks. In some embodiments, a dose of the ADC is administered to the subject about every 3 to 6 weeks. In other embodiments, a dose of the ADC is administered to the subject about every 3 weeks.

By “about” a certain number of weeks, is meant that number of weeks plus or minus 3 days. Thus a second dose administered about 2 weeks after the first dose may be administered 11 to 17 days after the first dose; a second dose administered about 3 weeks after the first dose may be administered 18 to 24 days after the first dose. In other embodiments, the ADC may be administered to the subject every 2, 3 or 4 weeks plus or minus 2 days, or plus or minus one day. While in an ideal scenario the doses might be administered to the subject exactly every 2, 3 or 4 weeks, for example, in practice this is not always possible, e.g. if a subject is unwell or unavailable on a given date, and therefore some flexibility in the dosing schedule is advantageous.

In particular embodiments, the ADC is administered about every 3 to 6 weeks, in a dosing schedule whereby the ADC is administered every 3 weeks, unless a delay is necessitated by a treatment-related adverse event, in which case a dose delay of up to 3 weeks is permitted.

When determining a dosing schedule, the date of each dose may be calculated from the date of the prior dose. That is to say, where the ADC is administered about every 3 weeks, the second dose is administered 18 to 24 days after the first dose, the third dose is administered 18 to 24 days after the second dose, and so on. Alternatively, the date of each dose may be calculated from the date of the first dose. In this instance, where the date of the first dose is defined as day 0, and doses are administered about every 3 weeks, the second dose would be administered between days 18 and 24, the third dose would be administered between days 39 and 45, and so on.

In some cases, the lengths between doses in a particular dosing schedule may be altered, if judged to be indicated by the subject's physician. For example the length of time between doses may be increased to reduce the level of side effects, or reduced for a greater effect, if considered appropriate.

As noted above, the subject treated with the ADC is administered at least one dose of the ADC of about 0.3 to about 3.4 mg/kg. Where the subject is administered more than one dose of the ADC, generally the same dosage is given each time. That said, the dosage may be increased or decreased if indicated, as judged by the subject's physician. For example the dosage may be decreased to reduce the level of side effects, or increased for a greater effect, if considered appropriate.

The dose, or each dose, administered to the subject, may be of about 0.6 to about 3.4 mg/kg, about 0.9 to about 3.4 mg/kg, about 1.2 to about 3.4 mg/kg, about 1.5 to about 3.4 mg/kg, about 1.8 to about 3.4 mg/kg, about 1.8 to about 3 mg/kg, about 2 to about 3.4 mg/kg, about 2 to about 3 mg/kg, about 2.2 to about 3.4 mg/kg or about 2.2 to about 3 mg/kg. In particular, the subject may be administered one of more doses of the ADC of about 1.8 to about 3.4 mg/kg, or about 2.2 to about 3 mg/kg.

In some embodiments, the subject may be administered one of more doses of the ADC of about 0.3 mg/kg, about 0.6 mg/kg, about 0.9 mg/kg, about 1.2 mg/kg, about 1.5 mg/kg, about 1.8 mg/kg, about 2.2 mg/kg, about 2.6 mg/kg, about 3 mg/kg or about 3.4 mg/kg. In particular, the subject may be administered one or more doses of the ADC of about 1.8 mg/kg, about 2.2 mg/kg, about 2.6 mg/kg, about 3 mg/kg or about 3.4 mg/kg.

In particular embodiments, the subject is administered a dose of the ADC of about 1.8 mg/kg, 2.2 mg/kg, 2.6 mg/kg, 3 mg/kg or 3.4 mg/kg, about every 3 weeks. By “about” a certain dosage value is meant the specified value+10%. All references to “about” a certain dosage specifically encompass the specified dosage, e.g. “about 2.2 mg/kg” specifically includes the value of 2.2 mg/kg.

As set out above, the dosages of the ADC used herein are defined as mg/kg. The ‘kg’ here refers to the body mass of the subject to be treated in kilograms. Thus for example, a dosage of 2.2 mg/kg means 2.2 mg per kg of the body mass of the subject. For example, a subject weighing 70 kg and receiving a dosage of 2.2 mg/kg would receive 154 mg of the ADC.

As noted above, the subject is generally administered more than one dose of the ADC, e.g. at least 2, 3, 4, 5, 6, 7, 8, 9, 10, 12, 14, 15, 16, 18 or 20 doses. For example, in particular embodiments, the subject is administered at least 5, 10, or 15 doses. The number of doses referred to here is the total number of doses administered to the subject through a course of therapy.

The subject may be administered the ADC for a duration of about 1 to 12 months. That is to say, the course of treatment with the ADC may last for about 1 to 12 months, i.e. the final dose of the ADC may be administered to the subject about 1 to 12 months after the first dose. For example, the subject may be administered the ADC for a duration of about 1 to 3, 1 to 4, 1 to 5, 1 to 6, 1 to 7, 1 to 8, 1 to 9, 1 to 10, 1 to 11, 2 to 3, 2 to 4, 2 to 5, 2 to 6, 2 to 7, 2 to 8, 2 to 9, 2 to 10, 2 to 11, 2 to 12, 3 to 4, 3 to 5, 3 to 6, 3 to 7, 3 to 8, 3 to 9, 3 to 10, 3 to 11, 3 to 12, 4 to 5, 4 to 6, 4 to 7, 4 to 8, 4 to 9, 4 to 10, 4 to 11, 4 to 12, 5 to 6, 5 to 7, 5 to 8, 5 to 9, 5 to 10, 5 to 11, 5 to 12, 6 to 7, 6 to 8, 6 to 9, 6 to 10, 6 to 11, 6 to 12, 7 to 8, 7 to 9, 7 to 10, 7 to 11, 7 to 12, 8 to 9, 8 to 10, 8 to 11, 8 to 12, 9 to 10, 9 to 11 or 9 to 12 months. In some cases the duration of treatment may be longer that 12 months, longer than 15 months or even longer than 18 months. The duration of treatment will vary between patients, with treatment being commenced and halted at the appropriate time for each subject, as determined by their physician. The longer the duration of treatment with the ADC, the more doses will be administered to the subject.

Generally, the course of treatment will last until confirmed disease progression in the subject, unacceptable toxicity to the subject or death of the subject. Confirmed disease progression may indicate that the ADC is not, or is no longer, effective in treating the subject's cancer. Toxicity of the ADC to the subject may be considered unacceptable if the negative impact of the treatment side effects is greater than its anti-cancer benefit. In any event, confirmed disease progression or unacceptable toxicity can be readily determined by the subject's physician.

The length of the course of treatment (e.g. the time until confirmed disease progression, unacceptable toxicity or death) may be dependent on the type of cancer suffered by the subject, the stage of the cancer at the beginning of therapy and/or the line of treatment for which the ADC is used. For example, on average, treatment of subjects with gastric cancer is likely to last for longer than treatment of subjects with pancreatic cancer. Subjects with pancreatic cancer may be treated with the ADC for a duration of about 1 to 12, 1 to 11, 1 to 10, 1 to 9, 1 to 8, 1 to 7, 1 to 6, 1 to 4, 1 to 3, 2 to 4 or 2 to 3 months, though as set out above any individual subject may receive treatment with the ADC for longer (or indeed shorter) than this, if appropriate, depending on the overall health of the patient and their response to the treatment.

Subjects with gastric cancer or GEJ cancer may be treated with the ADC for a duration of about 3 to 9, 3 to 8, 3 to 7, 3 to 6, 4 to 8 or 4 to 6 months, though again, any individual subject may receive treatment with the ADC for longer (or indeed shorter) than this, if appropriate, depending on the overall health of the patient and their response to the treatment.

Generally, the length of the course of treatment (e.g. the time until confirmed disease progression, unacceptable toxicity or death) may also be dependent on how early in the course of the disease the ADC is administered to the subject. The earlier in treatment that the ADC is administered to the subject, the longer the treatment with the ADC will generally last. That is to say, where the ADC is administered as first line treatment, the treatment course is likely to last longer than where the ADC is administered as a second line treatment, and where the ADC is administered as a second line treatment, the treatment course is likely to last longer than where the ADC is administered as a third line treatment, etc. For example, where the ADC is administered as a first line treatment, the subject may be treated with the ADC for a duration of about 3 to 12, 3 to 11, 3 to 10, 3 to 9, 3 to 8, 3 to 7, 3 to 6, 4 to 12, 4 to 11, 4 to 10, 4 to 9, 4 to 8, 4 to 7, 4 to 6, 6 to 12, 6 to 9 or 6 to 8 months. Where the ADC is administered as a second line treatment, the subject may be treated with the ADC for a duration of e.g. about 1 to 9, 1 to 8, 1 to 7, 1 to 6, 1 to 5, 1 to 4, 2 to 9, 2 to 8, 2 to 7, 2 to 6, 2 to 5, 2 to 4, 3 to 9, 3 to 8, 3 to 7, 3 to 6, 4 to 9, 4 to 8, 4 to 7 or 4 to 6 months.

Indeed, the length of treatment duration is likely to be impacted by the combination of the line of treatment as which the ADC is used and the type of cancer to be treated, e.g. first line of treatment of gastric cancer is likely to have a much longer duration that second or subsequent line treatment of pancreatic cancer.

As shown in the examples below, treatment of cancer with the dosages of the ADC described above has been found to be effective. For example, in a given population of patients with gastric or GEJ cancer, the overall response rate (ORR, alternatively referred to as the objective response rate) may be at least 25%, 30%, 35%, 40%, 45% or 50%. The ORR is defined as the proportion of patients showing either a complete response (CR) or partial response (PR) to the treatment, according to RECIST classification (e.g. RECIST version 1.1, Eisenhauer et al., European Journal of Cancer 45:228-247, 2009). This may particularly be the case when the ADC is administered as a second or further line of therapy (i.e. in subjects who have already received at least one prior line of therapy).

In particular, treatment of subjects with gastric or GEJ cancer with the ADC described herein as a second or further line of treatment, at a dosage of 2.2 mg/kg to 3 mg/kg about every 3 weeks, may have an ORR of at least 25%, 30%, 35%, 40%, 45% or 50%. More specifically, treatment of such subjects at a dosage of 2.2 mg/kg, 2.6 mg/kg or 3 mg/kg of the ADC about every 3 weeks may have an ORR of at least 25%, 30%, 35%, 40%, 45% or 50%.

Alternatively or additionally, in a given population of patients with gastric or GEJ cancer, the disease control rate (DCR) may be at least 50%, 55%, 60%, 65%, 70%, 75% or 80%. The disease control rate is defined as the proportion of patients showing a complete response (CR), partial response (PR) or stable disease (SD) to the treatment, according to RECIST classification (e.g. RECIST version 1.1, Eisenhauer et al., supra). This may particularly be the case when the ADC is administered as a second or further line of therapy (i.e. in subjects who have already received at least one prior line of therapy).

In particular, treatment of subjects with gastric or GEJ cancer with the ADC described herein as a second or further line of treatment, at a dosage of 2.2 mg/kg to 3 mg/kg about every 3 weeks, may have a DCR of at least 50%, 55%, 60%, 65%, 70%, 75% or 80%. More specifically, treatment of such subjects at a dosage of 2.2 mg/kg, 2.6 mg/kg or 3 mg/kg of the ADC about every 3 weeks may have a DCR of at least 50%, 55%, 60%, 65%, 70%, 75% or 80%.

Alternatively or additionally, in a given population of patients with gastric or GEJ cancer, treatment with the ADC described herein may result in a median progression-free survival of at least 3, 3.5, 4, 4.5 or 5 months. This may particularly be the case when the ADC is administered as a second or further line of therapy (i.e. in subjects who have already received at least one prior line of therapy).

In particular, treatment of subjects with gastric or GEJ cancer with the ADC described herein as a second or further line of treatment, at a dosage of 2.2 mg/kg to 3 mg/kg about every 3 weeks, may result in a median progression-free survival of at least 3, 3.5, 4, 4.5 or 5 months. More specifically, treatment of such subjects at a dosage of 2.2 mg/kg, 2.6 mg/kg or 3 mg/kg of the ADC about every 3 weeks may result in a median progression-free survival of at least 3, 3.5, 4, 4.5 or 5 months.

Alternatively or additionally, in a given population of patients with gastric or GEJ cancer, treatment with the ADC described herein may result in a median overall survival of at least 8, 9, 10, 11 or 12 months. This may particularly be the case when the ADC is administered as a second or further line of therapy (i.e. in subjects who have already received at least one prior line of therapy).

In particular, treatment of subjects with gastric or GEJ cancer with the ADC described herein as a second or further line of treatment, at a dosage of 2.2 mg/kg to 3 mg/kg about every 3 weeks, may result in a median overall survival of at least 8, 9, 10, 11 or 12 months. More specifically, treatment of such subjects at a dosage of 2.2 mg/kg, 2.6 mg/kg or 3 mg/kg of the ADC about every 3 weeks may result in a median overall survival of at least 8, 9, 10, 11 or 12 months.

Progression-free survival (PFS) and overall survival (OS) as referred to herein are counted from the beginning of treatment with the ADC.

The ADC described herein may be administered in the context of a monotherapy, by which is meant that the ADC is not administered in combination with any other anti-cancer drug. That is to say, when the ADC is administered as a monotherapy, no other anti-cancer drug is administered to the subject during the course of therapy using the ADC.

Alternatively, the ADC described herein may be administered as part of a combination therapy with one or more other anti-cancer drugs, such as a chemotherapy drug or an immunotherapy drug. The ADC described herein may be administered in combination with other modes of cancer treatment, such as radiotherapy or surgery. For instance, the ADC may be administered to a subject prior to surgery (i.e. as a neoadjuvant), e.g. for the purpose of shrinking a tumour to improve the prospects of surgical treatment. The ADC can also be used as adjuvant therapy following surgery, or in combination with surgery where resectable tumours are surgically removed and unresectable tumours are treated with the ADC.

Administration of the ADC to the subject may be by any suitable route. Generally the ADC is administered parenterally, e.g. intravenously, intramuscularly, topically or subcutaneously. Generally, the ADC is administered intravenously.

### Compositions

The ADC for use herein is generally administered in the context of a pharmaceutical composition. A pharmaceutical composition may comprise at least one pharmaceutically acceptable diluent, carrier or excipient, in addition to the ADC. The term “pharmaceutically acceptable” as used herein refers to ingredients that are compatible with other ingredients of the compositions as well as physiologically acceptable to the recipient. The nature of the composition and carriers or excipient materials may be selected in routine manner.

The pharmaceutical composition may be in any form known in the art, but may particularly be a liquid solution of the ADC. Suitable pharmaceutically acceptable diluents, carriers and excipients for inclusion in such liquid solutions are well known in the art. For instance, suitable diluents, carriers and excipients include sucrose, lactose, trehalose, glucose (and other sugars), polyols, liposomes, polyvinyl alcohol, mannitol, gelatin and alcohols.

Liquid pharmaceutical compositions, whether they be solutions, suspensions or other like form, may include one or more of the following: sterile diluents such as water for injection, Ringer's solution, isotonic sodium chloride, polyethylene glycols, glycerin, propylene glycol or other solvents; antibacterial agents such as benzyl alcohol or methyl paraben; antioxidants such as ascorbic acid or sodium bisulfite; chelating agents such as EDTA; buffers such as acetates, citrates or phosphates and agents for the adjustment of tonicity such as dextrose. A parenteral preparation can be enclosed in ampoules, disposable syringes or multiple dose vials made of glass or plastic.

The therapies disclosed herein are exemplified in the non-limiting figures and examples below.

### FIGURE LEGENDS

FIGS. 1A and 1B show the efficacy of CMG901 in the dose escalation phase of the trial (Part A, n=27). FIG. 1A shows the change in target lesion size from baseline;

FIG. 1B shows responses and treatment duration. Measurable lesions were only present in 24/27 patients. Horizontal dashed lines represent 20% increase and 30% reduction in tumour size. CR=complete response; PR=partial response; SD=stable disease, PD=progressive disease; NE—not evaluable.

FIG. 2 shows the efficacy of CMG901 in the subgroup with CLDN18.2-positive gastric/GEJ cancer (n=89). The graph shows the best percentage change from baseline in target lesion size in subgroup with CLDN18.2-positive G/GEJ cancer. Each bar represents an individual patient. Measurable lesions were only present in 89/93 patients receiving CMG901 at doses of 2.2, 2.6, and 3.0 mg/kg. Horizontal dashed lines represent 20% increase and 30% reduction in tumour size. G/GEJ=gastric/gastroesophageal junction; CR=complete response; PR=partial response; SD=stable disease; PD=progressive disease; NE=not evaluable.

FIG. 3 shows the response and duration of treatment of CMG901 in the subgroup with CLDN18.2-positive G/GEJ cancer (n=93). G/GEJ=gastric/gastroesophageal junction; CR=complete response; PR=partial response; SD=stable disease; PD=progressive disease.

FIGS. 4A and 4B show the survival outcomes for patients treated with CMG901 in subgroup with CLDN18.2-positive G/GEJ cancer (n=93). FIG. 4A shows the PFS data and FIG. 4B shows OS data in a subgroup of gastric/GEJ cancer patients with CLDN18.2 expression of ≥2+in ≥20% of tumour cells (2.0, 2.6, and 3.0 mg/kg doses; n=93). G/GEJ=gastric/gastroesophageal junction; PFS=progression-free survival; OS=overall survival.

FIG. 5 shows subgroup analyses in patients with CLDN18.2-positive G/GEJ cancer. Panel A shows the confirmed objective response rate (ORR). Panel B shows progression-free survival (PFS). Dotted lines represent the ORR or median PFS for the overall study population receiving CMG901 at doses of 2.0, 2.6, and 3.0 mg/kg (33% for confirmed ORR and 4.8 months for median PFS). G/GEJ=gastric/gastroesophageal junction; ECOG PS=Eastern Cooperative Oncology Group performance status; PD-1=programmed cell death protein 1.

FIGS. 6A and 6B show the efficacy of CMG901 in G/GEJ cancer patients. FIG. 6A shows the best percentage change from baseline in target lesion size. Measurable lesions were only present in 109/113 patients receiving CMG901 at doses of 2.0, 2.6, and 3.0 mg/kg. Horizontal dashed lines represent 20% increase and 30% reduction in tumor size. FIG. 6B shows the response and duration of treatment of CMG901 in all 113 patients. G/GEJ=gastric/gastroesophageal junction; CR=complete response; PR=partial response; SD=stable disease; PD=progressive disease; NE=not evaluable.

FIGS. 7A and 7B show the survival outcomes of CMG901 in G/GEJ cancer patients. FIG. 7A shows progression-free survival (PFS), while FIG. 7B shows the overall survival (OS). G/GEJ=gastric/gastroesophageal junction; PFS=progression-free survival; OS=overall survival.

FIG. 8 shows subgroup analyses of G/GEJ cancer patients. Panel A shows the objective response rate (ORR). Panel B shows progression-free survival (PFS). Dotted lines represent the ORR or PFS for the overall study population receiving CMG901 at doses of 2.0, 2.6, and 3.0 mg/kg (28% for confirmed ORR and 3.7 months for median PFS). G/GEJ=gastric/gastroesophageal junction; ECOG PS=Eastern Cooperative Oncology Group performance status; PD-1=programmed cell death protein 1.

### EXAMPLES

### Background

CMG901 is a potential first-in-class CLDN18.2 specific antibody-drug conjugate (ADC) that consists of a humanized anti-CLDN18.2 immunoglobulin G1 antibody (CM311) attached via a protease-cleavable linker to the highly cytotoxic microtubule-disrupting agent monomethyl auristatin E (MMAE). Preclinical studies showed that CMG901 induced potent anti-tumour activity in gastric and pancreatic patient-derived xenograft (PDX) models (WO 2022/078523). To evaluate the safety/tolerability, preliminary efficacy, pharmacokinetics and immunogenicity of CMG901 in patients with advanced gastric/GEJ cancer, pancreatic cancer and other solid tumours, a phase 1 trial was conducted. Here, the findings from the overall population in the dose escalation phase (Part A), and from the gastric/GEJ cancer cohort in the dose expansion phase (Part B), are reported.

### Methods

### Trial Design

KYM901 (NCT04805307) is a multicentre, phase 1 trial conducted in patients with advanced solid tumours. This trial included dose escalation (Part A: 0.3-3.4 mg/kg) and dose expansion (Part B: 2.2, 2.6 and 3.0 mg/kg) phases.

Part A consisted of eight dose-escalation cohorts, with the initial two CMG901 dose levels following an accelerated titration design, while the subsequent six dose levels followed traditional 3+3 dose escalation design. The planned sequential dose escalations were 0.3, 0.6, 1.2, 1.8, 2.2, 2.6, 3.0 and 3.4 mg/kg. In Part B, patients received CMG901 at assigned doses of 2.2, 2.6 and 3.0 mg/kg. CMG901 was administrated intravenously once every 3 weeks until confirmed disease progression, unacceptable toxicity, initiation of new anti-tumour therapy, withdrawal from the study, or death, whichever occurred first. Dose delay due to treatment-related adverse events (AEs) was permitted up to 3 weeks after the planned date of infusion.

### Trial Procedures

The dose-escalation scheme of CMG901 was guided by the safety review committee based on evidence of all dose-limiting toxicities (DLTs) occurring within 21 days post CMG901 first infusion. A DLT was defined as a CMG901-related occurrence of any pre-described adverse events (AEs), details of which were set out in the protocol (available at clinicaltrials.gov). If one patient in each cohort developed a DLT during the accelerated titration, the dose was switched to the traditional 3+3 dose-escalation. If one of 3 patients in each cohort developed a DLT during the traditional 3+3 dose escalation, then an additional 3 patients were to be treated at that dose level.

Blood samples were collected at protocol-specified timepoints for analyses of complete blood count, blood biochemistry, pharmacokinetics (after first and third infusions), and immunogenicity. Tumour responses were evaluated by computed tomography or magnetic resonance imaging of the chest, abdomen and pelvis using Response Evaluation Criteria in Solid Tumors (RECIST) version 1.1 by investigators at the end of week 3, every 6 weeks thereafter, and every 12 weeks after 1 year until radiological progression of disease, initiation of new anti-tumour therapy, withdrawal from the study or death, whichever occurred first.

AEs were monitored during the trial until the end of the safety follow-up (up to 4 weeks after the last administration), initiation of new anti-tumour therapy, withdrawal from the study or death, whichever occurred first. The severity of AEs was graded using Common Terminology Criteria for Adverse Events (CTCAE) v5.0.

### Sample Size

The sample size for the dose-escalation phase (Part A) was determined using a modified 3+3 design, and additional patients could be enrolled to account for withdrawals that were unrelated to toxicity. For the dose-expansion phase (Part B), it was planned to enroll 50-150 patients for treatment in three cohorts (2.2, 2.6, and 3.0 mg/kg).

### Patients

Eligibility criteria included individuals aged 18 or older, with a diagnosis of advanced gastric/GEJ cancer, pancreatic cancer or other solid tumours, who demonstrated refractoriness and/or intolerance to standard therapies, and had an Eastern Cooperative Oncology Group (ECOG) performance status score of ≤1, a life expectancy of >3 months, and either evaluable lesions (Part A) or a minimum of one measurable lesion (Part B) as per the Response Evaluation Criteria in Solid Tumors version 1.1 (RECIST v1.1). CLDN18.2 expression was retrospectively assessed in Part A (as described below). For patient enrollment in Part B, a requirement of moderate-to-strong CLDN18.2 expression (defined as ≥2 intensity) in ≥5% tumour cells for gastric/GEJ cancer was required. Patients were excluded if they had a known allergy to monoclonal antibodies or had received any anti-tumour treatments within 28 days. Full eligibility criteria are listed in the trial protocol.

### Determination of CLDN18.2 Expression

To detect CLDN18.2 expression, immunohistochemistry with an anti-CLDN18.2 antibody kit (either Abcam catalogue #222512 or Abcam catalogue #241330) was performed to assess the archival or fresh tissue specimens obtained prior to CMG901 treatment. Tumour tissue sections were fixed with 4% formaldehyde solution and incubated with different antibodies at the manufacturer-specified concentrations at 4° C. overnight, followed by incubation with HRP-conjugated secondary antibody at room temperature for 1 h. Chromogenic 3,3′-diaminobenzidine substrate was added to visualize the expression of CLDN18.2.

### Endpoints

The primary endpoints for Part A were safety/tolerability and determination of the maximum tolerated dose (MTD). The primary endpoints for Part B were the objective response rate (ORR) and identifying the recommended phase 2 dose (RP2D).

Secondary endpoints included disease control rate (DCR), duration of response (DOR), progression-free survival (PFS), overall survival (OS), and the correlation between the clinical response to CMG901 and CLDN18.2 expression. Additional secondary endpoints included pharmacokinetic parameters after single and multiple doses and anti-drug antibody (ADA) production.

### Study Oversight

The trial was conducted following the Declaration of Helsinki, the International Conference on Harmonization of Good Clinical Practice guidelines, and applicable regulatory requirements. The trial received the ethics committee approvals of each trial centre, and written informed consents were obtained from all patients.

### Statistical Analysis

DLT analyses were performed on DLT-evaluable patients (applicable only for Part A). The full analysis set included patients who received at least one administration of CMG901. The efficacy set comprised all gastric/GEJ cancer patients from Part A and Part B who received CMG901 at doses of 2.2, 2.6, and 3.0 mg/kg at least once, and for whom post-treatment imaging evaluation results were available. Additionally, a subgroup of patients selected from the efficacy set, who had detectable CLDN18.2 expression, were included in the biomarker set. The safety set, pharmacokinetic concentration set, pharmacokinetic analysis set and immunogenicity set included all patients who received one or more doses of CMG901 and had at least one corresponding qualified result.

SAS v9.4 was used for statistical analyses. Categorical variables were summarized by frequencies and percentages. Time to event endpoints were analyzed using Kaplan-Meier methods, with a two-sided 95% confidence interval (CI) calculated. Subgroups based on patient characteristics were analyzed for ORR. Pharmacokinetic parameters of serum CMG901, total antibody and MMAE were determined using standard noncompartmental analysis (Phoenix WinNonlin v8.3).

### Results

### Trial Population

Between Dec. 24, 2020, and Jul. 24, 2023, 27 patients in Part A (13 for G/GEJ cancer and 14 for pancreatic cancer) and 149 patients in Part B (107 for G/GEJ cancer, 40 for pancreatic cancer and 2 for other solid tumours) were enrolled at 31 sites in China (Table 1A). In Part A, 70% of patients were ECOG score 1, and the median number of lines of prior therapies was 3 (range 1-5, Table 2). Among the 113 patients (including six from Part A) with G/GEJ cancer who received CMG901 at doses of 2.2, 2.6, and 3.0 mg/kg in this trial, the median prior lines of therapy was 2 (range 1-6) (Table 1A). 74% of patients had received prior anti-programmed cell death protein 1 (PD-1) or anti-programmed death ligand 1 (PD-L1) therapies, and 4% had received prior CLDN18.2-targeting therapy (Table 1A). At the data cut-off date Jul. 24, 2023, 93 of 113 patients (82%) had discontinued CMG901 treatment, with 64 (57%) discontinuing due to progressive disease and 14 (12%) discontinuing due to patient request.

Among the 47 patients with pancreatic cancer who received CMG901 at doses of 2.2, 2.6, and 3.0 mg/kg in this trial, 32 (68%) had received at least one previous line of therapy. Exactly half of these (16 patients) had received prior immune checkpoint inhibitor therapy (Table 1B).

### Safety

In Part A, the MTD was not reached. At 2.2 mg/kg, one patient experienced a dose-limiting toxicity (DLT) with grade 3 pancreatitis, but no subsequent cases of pancreatitis were identified. All 27 patients reported at least one treatment-emergent adverse event (TEAE). Drug-related grade ≥3 TEAEs occurred in 5 patients (19%) (Table 3). The most frequent TEAEs were vomiting, decreased appetite, proteinuria, and anaemia (Table 3; Table 4). Three patients had treatment discontinuation due to drug-related TEAEs: fatigue and pancreatitis. One death occurred due to grade 5 pneumonia, which was not considered drug related (Table 3).

Safety data were reported for the 113 patients with advanced G/GEJ cancer who received CMG901 at doses of 2.2-3.0 mg/kg and the 47 patients with PDAC who received the same doses. All patients had at least one TEAE. The most commonly reported TEAEs in the G/GEJ cancer group were anaemia (63%), vomiting (58%), hypoalbuminaemia (58%), weight decreased (56%), and nausea (55%) (Table 5). Drug related grade ≥3 TEAEs occurred in 54% (61/113) of patients in the G/GEJ cancer group (Table 3); neutrophil count decreased (19%), vomiting (9%), anaemia (7%), white blood cell count decreased (7%), and decreased appetite (6%) were the most frequent. Serious AEs were reported in 47% of patients in the G/GEJ cancer group (31% were considered drug related) (Table 3). Similar results were seen in the Part A+Part B pancreatic cancer group, where 38% of patients reported a serious AE, of which 32% were considered treatment-related. Three deaths occurred due to TEAEs, of which one (cerebral haemorrhage) was considered to be related to CMG901, while the other two patients died of unknown reasons. 15 patients (13%) had a TEAE leading to dose reduction, and CMG901-related AEs leading to treatment discontinuation occurred in nine patients (Table 3).

Among the 134 patients from Part A and Part B (G/GEJ cancer), 25 (19%) had low grade peripheral neuropathy, with only three being classified as grade 2. The median time to the first onset of peripheral neuropathy was 2.1 months (range: 0.2-11.7). In addition, nine patients (8%) experienced CMG901-related infusion-related reaction, with three classified as grade 1, four as grade 2, and two as grade 4 (resulting in treatment discontinuation).

TABLE 1A
Patient demographics and baseline characteristics in G/GEJ cancer cohort (Full Analysis Set)

| | Part A: dose | | | | |
|---|---|---|---|---|---|
| | escalation | Part B: G/GEJ cancer cohort | | | |
| | Total | 2.2 mg/kg | 2.6 mg/kg | 3.0 mg/kg | Total |
| Characteristic | (n = 27) | (n = 44) | (n = 50) | (n = 19) | (n = 113*) |
| Age, years | 57.0 (48.0-63.0) | 55.0 (43.5-62.5) | 57.5 (46.0-67.0) | 53.0 (39.0-62.0) | 56.0 (44.0-64.0) |
| Sex^{#} | | | | | |
| Male | 14 (52%) | 23 (52%) | 26 (52%) | 10 (53%) | 59 (52%) |
| Female | 13 (48%) | 21 (48%) | 24 (48%) | 9 (47%) | 54 (48%) |
| ECOG PS | | | | | |
| 0 | 8 (30%) | 5 (11%) | 9 (18%) | 4 (21%) | 18 (16%) |
| 1 | 19 (70%) | 39 (89%) | 41 (82%) | 15 (79%) | 95 (84%) |
| Primary cancer type | | | | | |
| Gastric cancer | 12 (44%) | 42 (96%) | 42 (84%) | 18 (95%) | 102 (90%) |
| GEJ cancer | 1 (4%) | 2 (5%) | 8 (16%) | 1 (5%) | 11 (10%) |
| Pancreatic cancer | 14 (52%) | 0 | 0 | 0 | 0 |
| Claudin 18.2 expression | | | | | |
| of ≥2+ in ≥20% tumor cells | | | | | |
| in G/GEJ cancer cohort | | | | | |
| Yes | 8 (61%) | 32 (73%) | 45 (90%) | 16 (84%) | 93 (82%) |
| No | 4 (31%) | 11 (25%) | 5 (10%) | 3 (16%) | 19 (17%) |
| Missing | 1 (8%) | 1 (2%) | 0 | 0 | 1 (1%) |
| Median (range) lines | 3 (1-5) | 2 (1-5) | 2 (1-6) | 2 (1-5) | 2 (1-6) |
| of prior therapy | | | | | |
| Number of prior lines | | | | | |
| of therapy | | | | | |
| 1 | 2 (7%) | 14 (32%) | 20 (40%) | 9 (47%) | 43 (38%) |
| 2 | 11 (41%) | 18 (41%) | 13 (26%) | 7 (37%) | 38 (34%) |
| ≥3 | 14 (52%) | 12 (27%) | 17 (34%) | 3 (16%) | 32 (28%) |
| Prior anti-PD-1/PD-L1 | 16 (59%) | 33 (75%) | 38 (76%) | 12 (63%) | 83 (74%) |
| therapy | | | | | |
| Prior anti-claudin | 0 | 2 (5%) | 3 (6%) | 0 | 5 (4%) |
| 18.2 therapy | | | | | |

Data are median (IQR) or n (%).
*Includes 6 patients with G/GEJ cancer from Part A.
^{#}Sex was self-reported by the participants, with options “male” or “female”. G/GEJ = gastric/gastroesophageal junction; ECOG PS = Eastern Cooperative Oncology Group performance status; PD-1 = programmed cell death protein 1; PD-L1 = programmed death ligand 1.

TABLE 1B
Patient demographics and baseline characteristics in pancreatic cancer cohort

| | 2.2 mg/kg | 2.6 mg/kg | 3.0 mg/kg | Total |
|---|---|---|---|---|
| Characteristic | (n = 26) | (n = 13) | (n = 8) | (n = 47) |
| Age, years | 57.0 (42-73) | 60.0 (50-74) | 63.0 (47-70) | 59.0 (42-74) |
| ECOG PS | | | | |
| 0 | 1 (3.8%) | 1 (7.7%) | 2 (25.0%) | 4 (8.5%) |
| 1 | 25 (96.2%) | 12 (92.3%) | 6 (75.0%) | 43 (91.5%) |
| M stage at screening | | | | |
| M0 | 2 (7.7%) | 0 | 0 | 2(4.3) |
| M1 | 24 (92.3%) | 13 (100%) | 8(100) | 45 (95.7) |
| Unknown | 0 | 0 | 0 | 0 |
| No. of prior therapies | | | | |
| 1 | 8 (30.8%) | 3 (23.1%) | 2 (25.0%) | 13 (27.7%) |
| ≥2 | 9 (34.6%) | 7 (53.8%) | 3 (37.5%) | 19 (40.4%) |
| Types of prior therapies | | | | |
| Prior ICIs | 7 (29.6%) | 6 (46.2%) | 3 (37.5%) | 16 (34.0%) |
| Prior CLDN18.2 agent | 0 | 0 | 0 | 0 |

Data are median (range) or n (%). ECOG PS = Eastern Cooperative Oncology Group performance status; ICI = immune checkpoint inhibitor.

TABLE 2
Patient Demographics and Baseline Characteristics
in Dose-escalation Phase (Full Analysis Set)

| | 0.3 mg/kg | 0.6 mg/kg | 1.2 mg/kg | 1.8 mg/kg | 2.2 mg/kg |
|---|---|---|---|---|---|
| Characteristic | (N = 1) | (N = 1) | (N = 3) | (N = 4) | (N = 6) |
| Median age (range) - yr | 45 | 66 | 56 (55-57) | 54 (48-57) | 61 (42-67) |
| Sex - no. (%) | | | | | |
| Male | 0 | 1 (100.0) | 1 (33.3) | 2 (50.0) | 3 (50.0) |
| Female | 1 (100.0) | 0 | 2 (66.7) | 2 (50.0) | 3 (50.0) |
| ECOG PS - no. (%) | | | | | |
| 0 | 0 | 0 | 2 (66.7) | 1 (25.0) | 0 |
| 1 | 1 (100.0) | 1 (100.0) | 1 (33.3) | 3 (75.0) | 6 (100.0) |
| Cancer type - no. (%) | | | | | |
| Gastric/GEJ cancer | 0 | 0 | 1 (33.3) | 2 (50.0) | 3 (50.0) |
| Pancreatic cancer | 1 (100.0) | 1 (100.0) | 2 (66.7) | 2 (50.0) | 3 (50.0) |
| CLDN18.2 expression of ≥2+ | | | | | |
| in ≥20% tumour cells - no. (%) | | | | | |
| Yes | 0 | 0 | 2 (66.7) | 2 (50.0) | 0 |
| No | 1 (100) | 1 (100) | 1 (33.3) | 1 (25.0) | 5 (83.3) |
| Missing | 0 | 0 | 0 | 1 (25.0) | 1 (16.7) |
| Prior lines of therapy - no. (%) | | | | | |
| 1 | 0 | 1 (100) | 0 | 0 | 0 |
| 2 | 1 (100) | 0 | 1 (33.3) | 0 | 3 (50.0) |
| ≥3 | 0 | 0 | 2 (66.7) | 4 (100.0) | 3 (50.0) |
| Prior anti-PD-(L)1 therapy - no. (%) | 0 | 0 | 1 (33.3) | 2 (50.0) | 4 (66.7) |

| | 2.6 mg/kg | 3.0 mg/kg | 3.4 mg/kg | Total |
|---|---|---|---|---|
| Characteristic | (N = 3) | (N = 4) | (N = 5) | (N = 27) |
| Median age (range) - yr | 51 (44-63) | 54.5 (35-70) | 62 (31-70) | 57 (31-70) |
| Sex - no. (%) | | | | |
| Male | 2 (66.7) | 2 (50.0) | 3 (60.0) | 14 (51.9) |
| Female | 1 (33.3) | 2 (50.0) | 2 (40.0) | 13 (48.1) |
| ECOG PS - no. (%) | | | | |
| 0 | 1 (33.3) | 3 (75.0) | 1 (20.0) | 8 (29.6) |
| 1 | 2 (66.7) | 1 (25.0) | 4 (80.0) | 19 (70.4) |
| Cancer type - no. (%) | | | | |
| Gastric/GEJ cancer | 1 (33.3) | 2 (50.0) | 4 (80.0) | 13 (48.1) |
| Pancreatic cancer | 2 (66.7) | 2 (50.0) | 1 (20.0) | 14 (51.9) |
| CLDN18.2 expression of ≥2+ | | | | |
| in ≥20% tumour cells - no. (%) | | | | |
| Yes | 1 (33.3) | 2 (50.0) | 2 (40.0) | 9 (33.3) |
| No | 1 (33.3) | 1 (25.0) | 2 (40.0) | 13 (48.1) |
| Missing | 1 (33.3) | 1 (25.0) | 1 (20.0) | 5 (18.5) |
| Prior lines of therapy - no. (%) | | | | |
| 1 | 0 | 1 (25.0) | 0 | 2 (7.4) |
| 2 | 2 (66.7) | 2 (50.0) | 2 (40.0) | 11 (40.7) |
| ≥3 | 1 (33.3) | 1 (25.0) | 3 (60.0) | 14 (51.9) |
| Prior anti-PD-(L)1 therapy - no. (%) | 2 (66.7) | 2 (50.0) | 5 (100) | 16 (59.3) |

ECOG PS = Eastern Cooperative Oncology Group performance status; GEJ = gastroesophageal junction; PD-(L)1 = programmed cell death protein 1 or programmed death ligand 1.

TABLE 3
Safety and Tolerability of CMG901 (Safety Set)

| | | Gastric/GEJ |
|---|---|---|
| | Part A | cancer cohort |
| | (N = 27) | (N = 113*) |
| Any TEAE - no. (%) | 27 (100%) | 113 (100%) |
| Grade ≥3 | 11 (41%) | 72 (64%) |
| Drug-related | 26 (96%) | 112 (99%) |
| Grade ≥3 and drug-related | 5 (19%) | 61 (54%) |
| Any SAE - no. (%) | 8 (30%) | 53 (47%) |
| Drug-related | 4 (15%) | 35 (31%) |
| TEAEs leading to dose | 1 (4%) | 15 (13%) |
| reduction - no. (%) | | |
| Drug-related | 1 (4%) | 15 (13%) |
| TEAEs leading to treatment | 9 (33%) | 57 (50%) |
| interruption or delay - no. (%) | | |
| Drug-related | 5 (19%) | 33 (29%) |
| TEAEs leading to treatment | 6 (22%) | 9 (8%) |
| discontinuation - no. (%) | | |
| Drug-related | 3 (11%) | 9 (8%) |
| TEAEs leading to study | 1 (4%) | 3 (3%) |
| discontinuation - no. (%) | | |
| Drug-related | 0 | 1 (1%) |
| TEAEs leading to | 1 (4%) | 3 (3%) |
| death - no. (%) | | |
| Drug-related | 0 | 1 (1%) |

Data are presented as n (%).
*Includes 6 patients with G/GEJ cancer from dose-escalation phase (Part A). The maximum severity of TEAE was classified by Common Terminology Criteria for Adverse Events. TEAE = treatment-emergent adverse event. SAE = serious adverse events.

TABLE 4
Common treatment-emergent adverse events occurring in at least 15%
of the patients in the dose-escalation phase (Safety Set, n = 27).

| | 0.3 mg/kg | 0.6 mg/kg | 1.2 mg/kg | 1.8 mg/kg | 2.2 mg/kg | 2.6 mg/kg | 3.0 mg/kg | 3.4 mg/kg |
|---|---|---|---|---|---|---|---|---|
| | (N = 1) | (N = 1) | (N = 3) | (N = 4) | (N = 6) | (N = 3) | (N = 4) | (N = 5) |
| Haematological | | | | | | | | |
| Anaemia | 1 (100%) | 0 | 1 (33%) | 4 (100%) | 3 (50%) | 0 | 2 (50%) | 4 (80%) |
| Gastrointestinal | | | | | | | | |
| Vomiting | 0 | 1 (100%) | 2 (67%) | 2 (50%) | 5 (83%) | 2 (67%) | 3 (75%) | 4 (80%) |
| Nausea | 0 | 1 (100%) | 1 (33%) | 2 (50%) | 0 | 1 (33%) | 2 (50%) | 4 (80%) |
| Diarrhoea | 0 | 0 | 0 | 1 (25%) | 2 (33%) | 1 (33%) | 2 (50%) | 2 (40%) |
| Flatulence | 0 | 0 | 0 | 0 | 3 (50%) | 2 (67%) | 1 (25%) | 1 (20%) |
| Constipation | 0 | 0 | 2 (67%) | 1 (25%) | 0 | 0 | 1 (25%) | 1 (20%) |
| Other | | | | | | | | |
| Decreased appetite | 0 | 1 (100%) | 2 (67%) | 3 (75%) | 4 (67%) | 1 (33%) | 3 (75%) | 2 (40%) |
| Proteinuria | 0 | 0 | 2 (67%) | 2 (50%) | 4 (67%) | 2 (67%) | 2 (50%) | 4 (80%) |
| Aspartate aminotransferase increased | 1 (100%) | 0 | 1 (33%) | 2 (50%) | 2 (33%) | 1 (33%) | 3 (75%) | 3 (60%) |
| Weight decreased | 0 | 0 | 1 (33%) | 2 (50%) | 2 (33%) | 2 (67%) | 1 (25%) | 4 (80%) |
| Hypoalbuminaemia | 0 | 0 | 1 (33%) | 2 (50%) | 1 (17%) | 3 (100%) | 2 (50%) | 3 (60%) |
| Occult blood positive | 0 | 0 | 1 (33%) | 2 (50%) | 3 (50%) | 1 (33%) | 2 (50%) | 2 (40%) |
| White blood cell count decreased | 0 | 0 | 1 (33%) | 1 (25%) | 0 | 1 (33%) | 2 (50%) | 3 (60%) |
| Neutrophil count decreased | 0 | 0 | 0 | 1 (25%) | 0 | 1 (33%) | 2 (50%) | 4 (80%) |
| Alanine aminotransferase increased | 1 (100%) | 0 | 0 | 2 (50%) | 0 | 1 (33%) | 1 (25%) | 2 (40%) |
| Fatigue | 0 | 0 | 1 (33%) | 0 | 1 (17%) | 1 (33%) | 2 (50%) | 2 (40%) |
| Urinary occult blood positive | 0 | 0 | 0 | 1 (25%) | 1 (17%) | 0 | 2 (50%) | 2 (40%) |
| Asthenia | 0 | 0 | 2 (67%) | 2 (50%) | 0 | 0 | 1 (25%) | 1 (20%) |
| Hypoaesthesia | 0 | 0 | 0 | 1 (25%) | 0 | 1 (33%) | 2 (50%) | 2 (40%) |

Data are presented as n (%).

TABLE 5
Common treatment-emergent adverse events occurring in at least 15% of patients (Safety Set).

| | Part A: dose | | | | | | | | | |
|---|---|---|---|---|---|---|---|---|---|---|
| | escalation | | Part B: G/GEJ cancer cohort | | | | | | | |
| | Total (n = 27) | | 2.2 mg/kg (n = 44) | | 2.6 mg/kg (n = 50) | | 3.0 mg/kg (n = 19) | | Total (n = 113*) | |
| | Any | Grade ≥3 | Any | Grade ≥3 | Any | Grade ≥3 | Any | Grade ≥3 | Any | Grade ≥3 |
| Haematological | | | | | | | | | | |
| Anaemia | 15 (56%) | 1 (4%) | 27 (61%) | 6 (14%) | 31 (62%) | 8 (%) | 13 (68%) | 1 (5%) | 71 (63%) | 15 (13%) |
| Gastrointestinal | | | | | | | | | | |
| Vomiting | 19 (70%) | 1 (4%) | 23 (52%) | 5 (11%) | 30 (60%) | 4 (8%) | 12 (63%) | 2 (11%) | 65 (58%) | 11 (10%) |
| Nausea | 11 (41%) | 0 | 19 (43%) | 0 | 28 (56%) | 3 (6%) | 15 (79%) | 1 (5%) | 62 (55%) | 4 (4%) |
| Diarrhoea | 8 (30%) | 0 | 7 (16%) | 0 | 10 (20%) | 1 (2%) | 7 (37%) | 0 | 24 (21%) | 1 (1%) |
| Constipation | 5 (19%) | 0 | 6 (14%) | 0 | 10 (20%) | 0 | 5 (26%) | 0 | 21 (19%) | 0 |
| Abdominal pain | 3 (11%) | 0 | 5 (11%) | 0 | 7 (14%) | 2 (4%) | 8 (42%) | 1 (5%) | 20 (18%) | 3 (3%) |
| Flatulence | 7 (26%) | 0 | 1 (2%) | 0 | 2 (4%) | 0 | 0 | 0 | 3 (3%) | 0 |
| Other | | | | | | | | | | |
| Hypoalbuminaemia | 12 (44%) | 0 | 26 (59%) | 0 | 28 (56%) | 0 | 11 (58%) | 0 | 65 (58%) | 0 |
| Weight decreased | 12 (44%) | 0 | 18 (41%) | 0 | 30 (60%) | 2 (4%) | 15 (79%) | 1 (5%) | 63 (56%) | 3 (3%) |
| Neutrophil count | 8 (30%) | 2 (7%) | 18 (41%) | 5 (11%) | 28 (56%) | 11 (22%) | 13 (68%) | 5 (26%) | 59 (52%) | 21 (19%) |
| decreased | | | | | | | | | | |
| WBC count decreased | 8 (30%) | 0 | 17 (39%) | 4 (9%) | 28 (56%) | 4 (8%) | 12 (63%) | 0 | 57 (50%) | 8 (7%) |
| Decreased appetite | 16 (59%) | 0 | 19 (43%) | 6 (14%) | 27 (54%) | 1 (2%) | 10 (53%) | 1 (5%) | 56 (50%) | 8 (7%) |
| AST increased | 13 (48%) | 1 (4%) | 19 (43%) | 0 | 18 (36%) | 0 | 10 (53%) | 0 | 47 (42%) | 0 |
| Asthenia | 6 (22%) | 0 | 13 (30%) | 3 (7%) | 12 (24%) | 0 | 8 (42%) | 1 (5%) | 33 (29%) | 4 (4%) |
| Proteinuria | 16 (59%) | 0 | 10 (23%) | 0 | 11 (22%) | 1 (2%) | 9 (47%) | 0 | 30 (27%) | 1 (1%) |
| ALT increased | 7 (26%) | 1 (4%) | 14 (32%) | 0 | 11 (22%) | 0 | 6 (32%) | 0 | 31 (27%) | 0 |
| Hyponatraemia | 4 (15%) | 0 | 9 (21%) | 0 | 15 (30%) | 0 | 5 (26%) | 0 | 29 (26%) | 0 |
| Hypoaesthesia | 6 (22%) | 0 | 7 (16%) | 0 | 9 (18%) | 0 | 6 (32%) | 0 | 22 (20%) | 0 |

Data are presented as n (%). * Includes 6 patients with G/GEJ cancer from Part A. G/GEJ = gastric/gastroesophageal junction; WBC = white blood cell; AST = aspartate aminotransferase; ALT = alanine aminotransferase.

### Efficacy

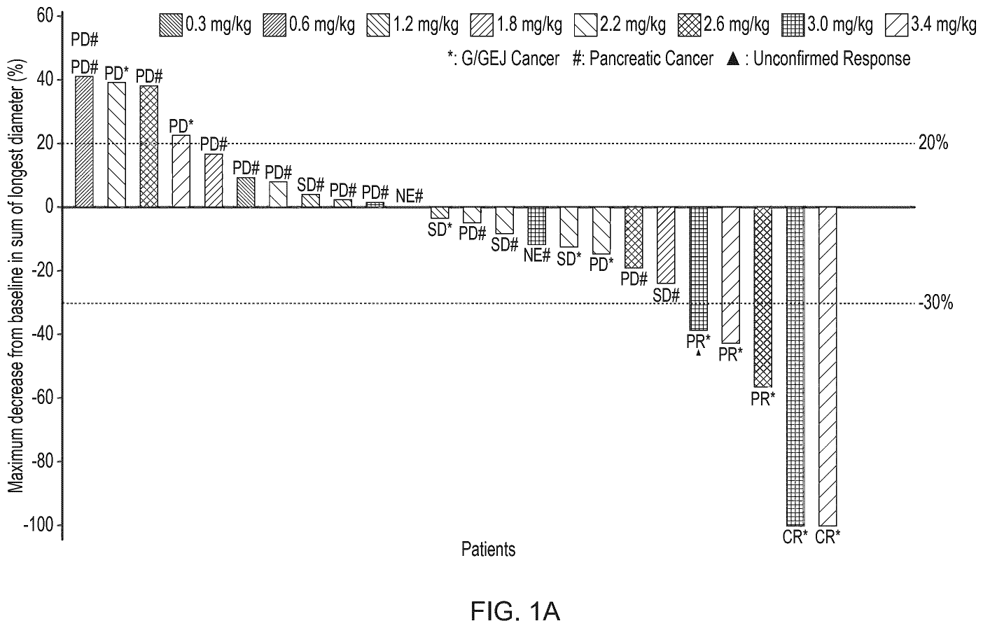

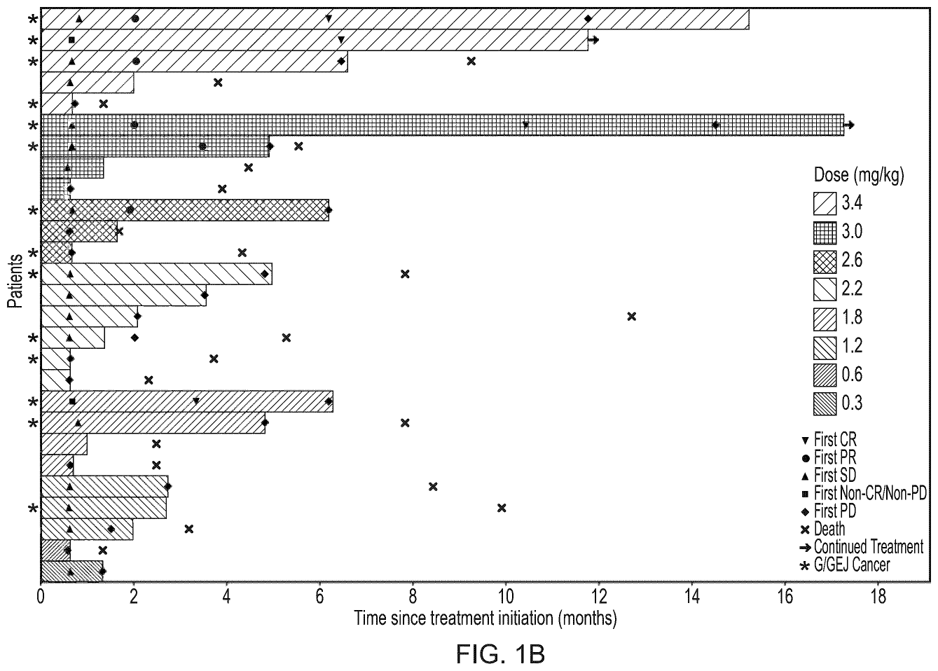

Anti-tumor activity was observed at doses from 1.8 mg/kg upwards. One patient with peritoneal metastasis in the 1.8 mg/kg cohort achieved complete response following 5 cycles of CMG901, with a PFS of 6.2 months. Detailed efficacy data of CMG901 in Part A is shown in FIGS. 1A and 1B.

For G/GEJ cancer patients with moderate-to-strong CLDN18.2 expression in <20%, ≥20%, ≥30%, ≥40%, ≥50% and ≥60% of tumour cells, confirmed ORRs were 5%, 33%, 34%, 36%, 36% and 36%, respectively. This suggested a possible correlation between CLDN18.2 expression and anti-tumor activity of CMG901. Therefore, we defined moderate-to-strong CLDN18.2 expression in ≥20% of tumor cells as CLDN18.2-positive, while anything below this threshold was not considered CLDN18.2-positive.

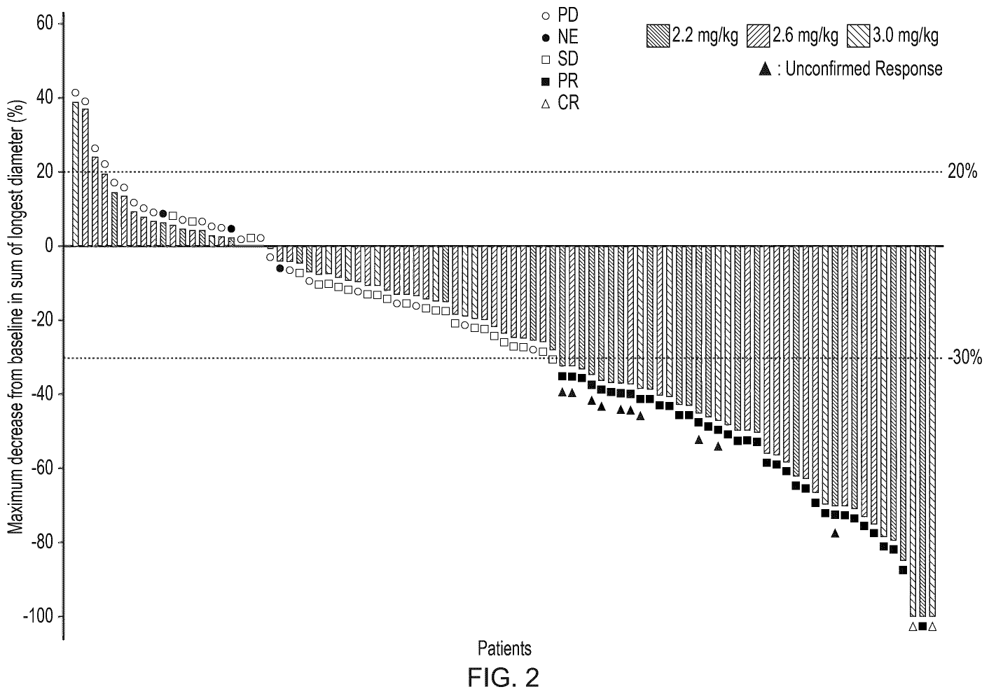

For 89 response-evaluable CLDN18.2-positive patients, the confirmed ORR was 33% (95% CI 23.0-43.3; including two complete responses), with an ORR of 42% in the 2.2 mg/kg cohort (Table 6). Confirmed disease control was seen in 62 patients (70%; 95% CI 59.0-79.0). The median time to response was 2.0 months (95% CI 2.1-3.5), and the median duration of response (DOR) was 5.7 months (95% CI 4.2-8.1) (Table 6). Tumour shrinkage (defined as reduction in sum of the longest diameters of the target lesions from baseline) is shown in FIG. 2. In the 3.0 mg/kg cohort, one patient, who had previously received a first-line combination of PD-1 inhibitor and chemotherapy, achieved a 100% reduction in the size of all target and non-target lesions following seven cycles of CMG901 administration.

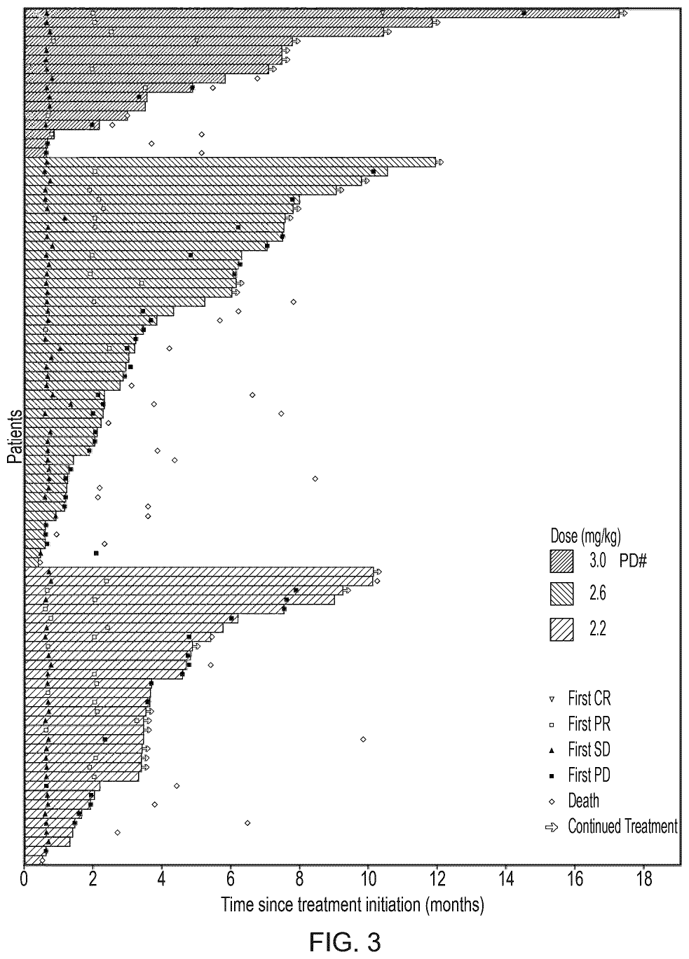

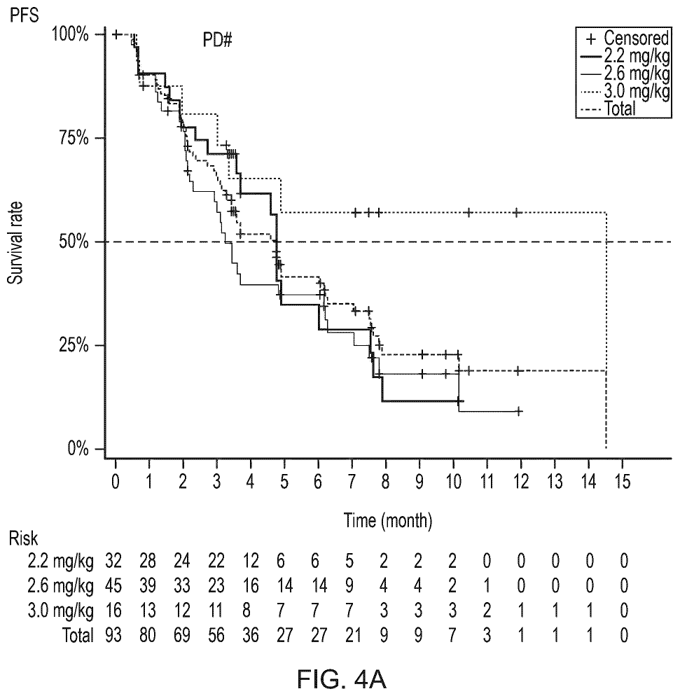

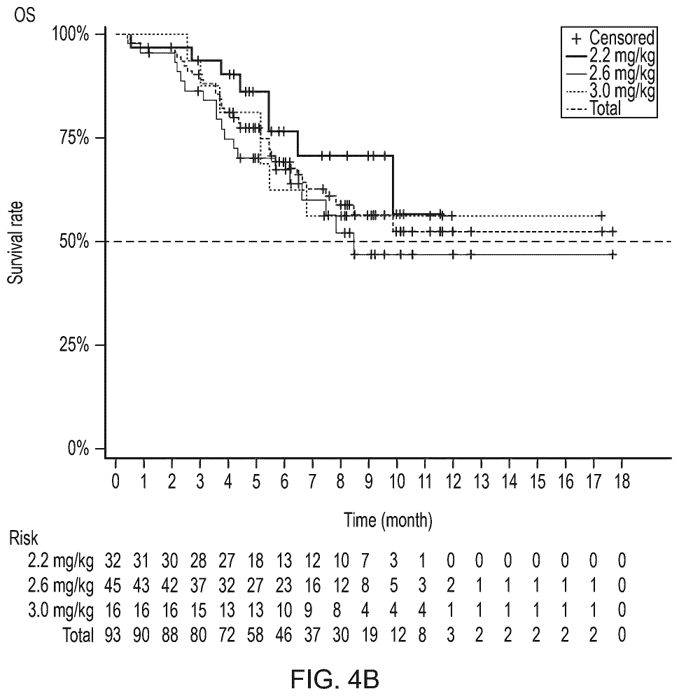

As of the data cut-off date, 23 of 93 CLDN18.2-positive patients (25%) continued CMG901 treatment (FIG. 3). After a median follow up of 6.0 months (IQR 4.2-8.3), the median PFS in the 2.2, 2.6, and 3.0 mg/kg cohorts were 4.8, 3.3, and 14.5 months, respectively (FIG. 4A). The median OS was not reached (95% CI 7.5—not reached [NR]) (Table 7; FIG. 4B). The estimated 6-month and 9-month OS rates were 69% (95% CI 58.2-78.1) and 56% (95% CI 43.6-67.3), respectively. Among patients who had received >2 prior lines of therapies, the median PFS was 4.8 months (95% CI 3.4-6.3), and the median OS was not reached (95% CI 6.8—NR), with a 9-month OS rate of 54%.

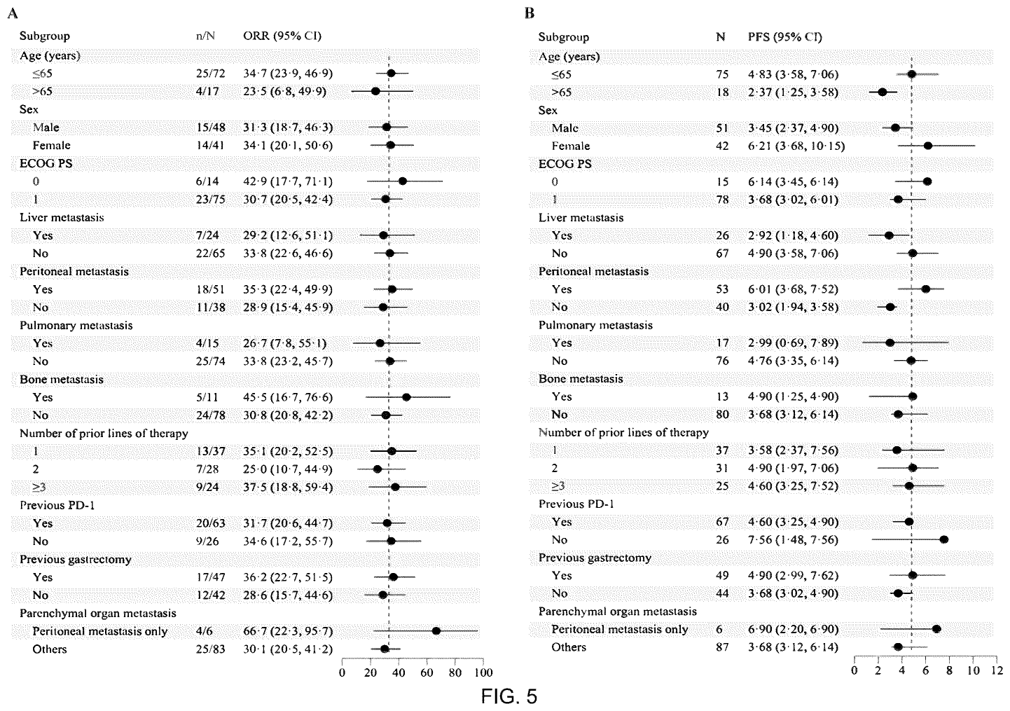

Subgroup analyses were conducted to explore the association between confirmed ORR or PFS and baseline characteristics in the 93 patients with CLDN18.2-positive G/GEJ cancer (FIG. 5). Findings indicated similar ORR and PFS irrespective the number of prior lines of therapy. 18 (35%) patients with peritoneal metastasis achieved a confirmed objective response, with a median PFS of 6.0 months.

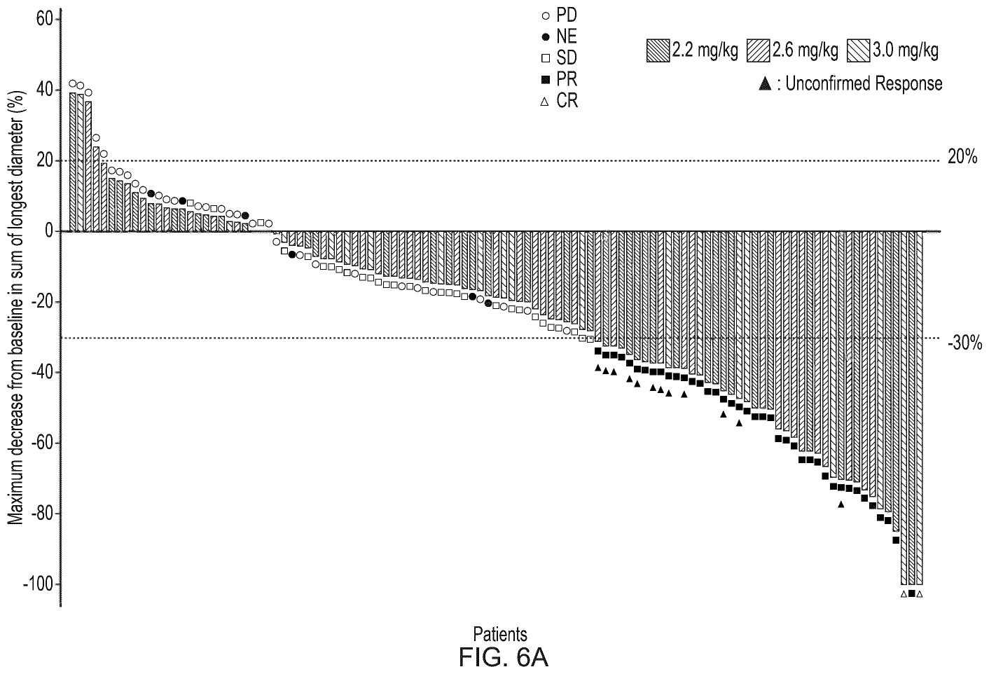

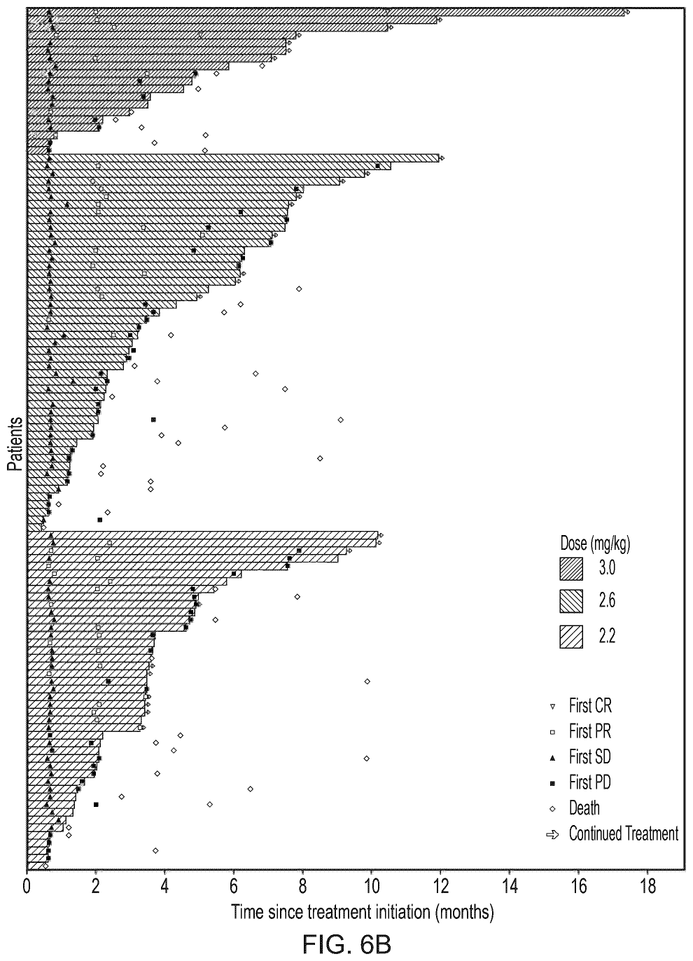

Anti-tumour responses were also assessed across the cohort, in 109 of 113 patients with advanced gastric/gastroesophageal junction (G/GEJ) cancer. 30 patients (28%; 95% CI 19.4-36.9) reached a confirmed objective response. Confirmed disease control rate was 65% (n=71, 95% CI 55.4-74.0) (FIG. 6A, Table 8). The median duration of response was 5.6 months (95% CI 4.2-8.1) and median time to response was 2.0 months (95% CI 2.2—not reached [NR]) (Table 8).

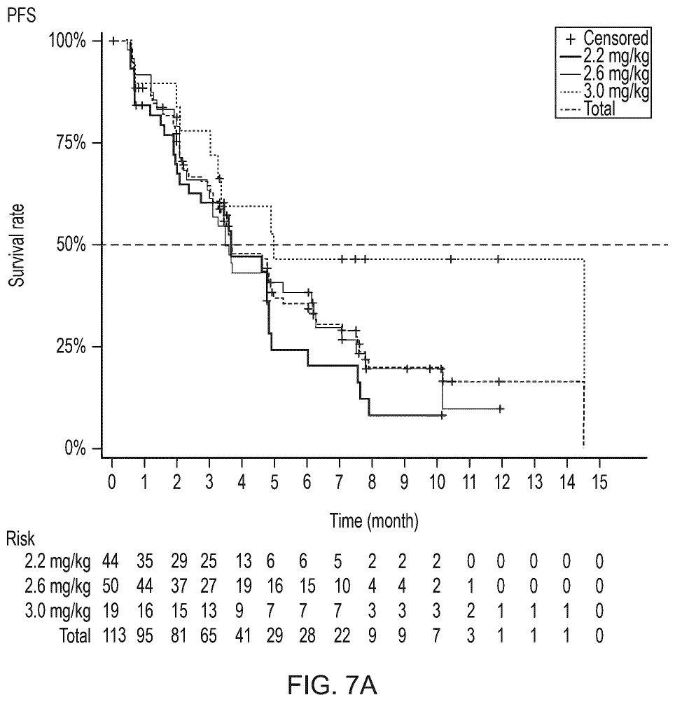

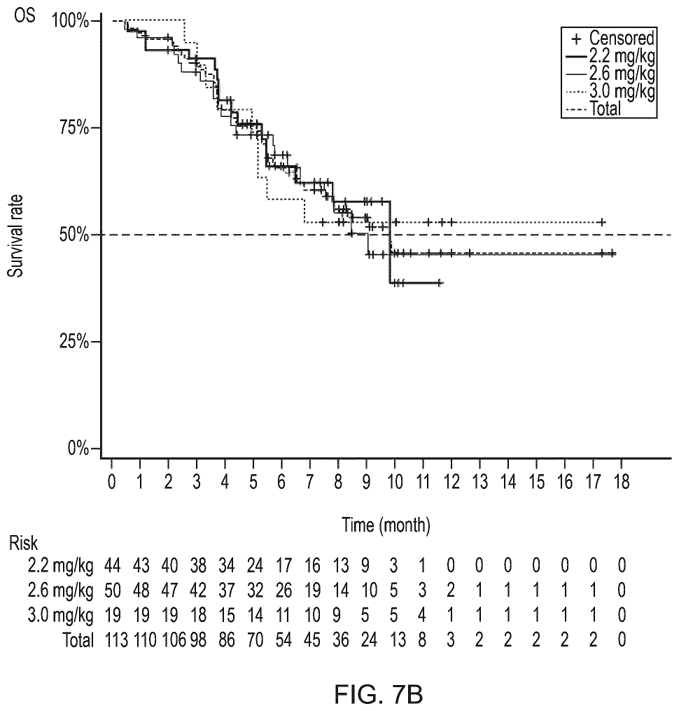

As of the data cut-off date, 26/113 patients with G/GEJ cancer (23%) continued treatment (FIG. 6B). With a median follow-up of 5.7 months (IQR, 4.2-8.5), the median progression-free survival was 3.7 months (95% CI 3.3-4.9) (FIG. 7A), and the median overall survival was 9.8 months (95% CI 6.8—NR) (Table 9; FIG. 7B).

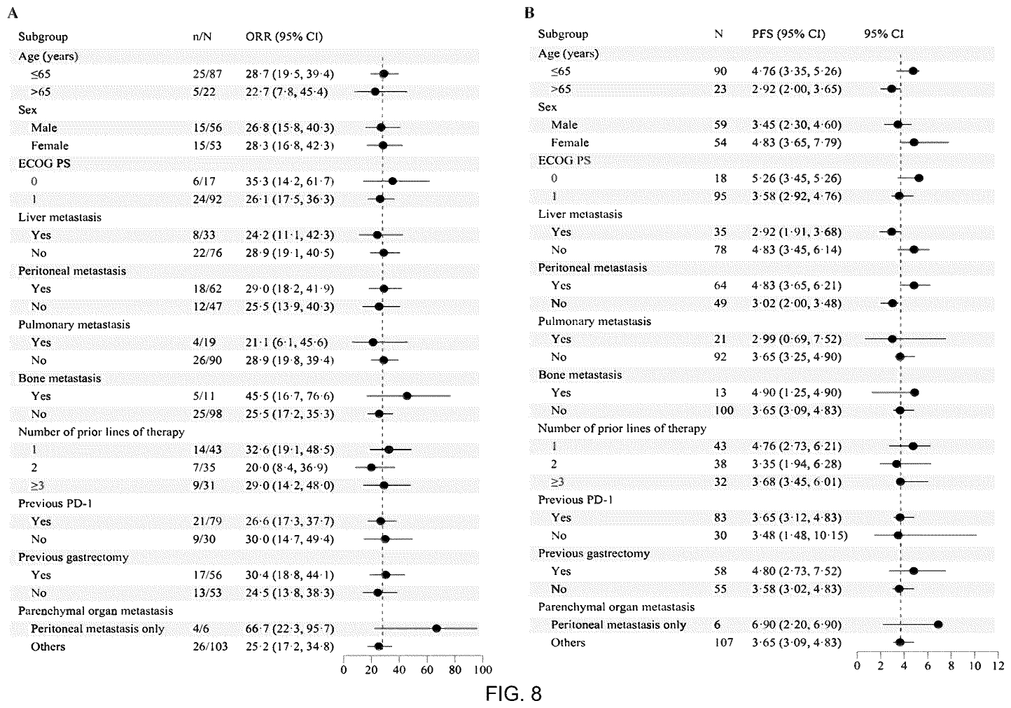

Subgroup analyses showed that ORR and PFS remained consistent regardless of the number of prior lines of therapy and previous anti-PD-1 therapy. In addition, a high ORR (4/6, 67%) and long median PFS (6.9 months) were observed in G/GEJ cancer patients with exclusively peritoneal metastasis (FIG. 8).

In patients with pancreatic cancer (pancreatic ductal adenocarcinoma, PDAC), confirmed responses were seen in 4/43 patients (9%), all of which were in patients with cancers containing ≥50% CLDN18.2-positive tumour cells, see Tables 10 and 11. Unconfirmed responses were seen in 5/43 patients (12%): 2/25 (8%) patients in the 2.2 mg/kg group, 1/12 (8%) patients in the 2.6 mg/kg group and 2/7 (29%) patients in the 3 mg/kg group.

In patients with cancers containing ≥50% CLDN18.2-positive tumour cells median PFS was 3.3 months. The median overall survival was not reached at the data cut-off (Table 12).

### Pharmacokinetics and Immunogenicity

The pharmacokinetics of CMG901 was assessed in the 134 G/GEJ cancer patients. The systemic exposure of CMG901 (defined as area under the concentration-time curve from time 0 to infinity in Cycle 1) showed a generally dose-proportional increase, while patients had typical low systemic exposure to unconjugated MMAE with the mean observed maximum concentration less than 10 ng/ml across all dose levels. 53 patients (40%) were confirmed to be positive for ADA: three had pre-existing ADA, one had treatment-boosted ADA, and 49 had treatment-induced ADA. The impact of ADA on efficacy and safety has not yet been analysed.

TABLE 6
Best overall response evaluation of CMG901 in patients with CLDN18.2-positive G/GEJ cancer (Efficacy Set)

| | 2.2 mg/kg | 2.6 mg/kg | 3.0 mg/kg | Total |
|---|---|---|---|---|
| | (n = 31) | (n = 42) | (n = 16) | (n = 89*) |
| BOR | | | | |
| CR | 0 | 0 | 2 (13%) | 2 (2%) |
| PR^{#} | 19 (61%) | 12 (29%) | 6 (38%) | 37 (42%) |
| SD | 3 (10%) | 16 (38%) | 5 (31%) | 24 (27%) |
| PD | 7 (23%) | 13 (31%) | 3 (19%) | 23 (26%) |
| NE | 2 (7%) | 1 (2%) | 0 | 3 (3%) |
| ORR | 19 (61%; 42.2-78.2) | 12 (29%; 15.7-44.6) | 8 (50%; 24.7-75.3) | 39 (44%; 33.3-54.7)⁺ |
| Confirmed ORR | 13 (42%; 24.5-60.9) | 10 (24%; 12.1-39.5) | 6 (38%; 15.2-64.6) | 29 (33%; 23.0-43.3) |
| DCR | 22 (71%; 52.0-85.8) | 28 (67%; 50.5-80.4) | 13 (81%; 54.4-96.0) | 63 (71%; 60.2-79.9) |
| Confirmed DCR^{δ} | 22 (71%; 52.0-85.8) | 28 (67%; 50.5-80.4) | 12 (75%; 47.6-92.7) | 62 (70%; 59.0-79.0) |
| Time to response | | | | |
| Number | 19 | 12 | 8 | 39 |
| Median (95% CI), months | 2.0 (2.0-2.4) | 2.1 (2.3-NR) | 2.0 (0.9-NR) | 2.0 (2.1-3.5) |
| Duration of response^{#} | | | | |
| Number | 19 | 12 | 8 | 39 |
| Median (95% CI), months | 5.3 (2.6-7.2) | 5.7 (2.9-NR) | 12.6 (1.5-NR) | 5.7 (4.2-8.1) |

Data are presented as n (%) unless stated otherwise. Best responses of each patient were assessed according to RECIST v1.1 by the investigator.
*Includes 3 patients with G/GEJ cancer from the dose-escalation phase.
^{#}Includes two patients who had the potential to achieve a confirmed PR.
^{δ}Calculated as the proportion of patients showing confirmed CR, PR, or SD for a minimum of 5 weeks from the first dosing date.
⁺10 partial responders had unconfirmed partial responses. G/GEJ = gastric/gastroesophageal junction; BOR = best overall response; CR = complete response; PR = partial response; SD = stable disease; PD = progressive disease; NE = not evaluable; ORR (CR + PR) = objective response rate; CI = confidence interval; DCR (CR + PR + SD) = disease control rate; NR = not reached.

TABLE 7
Efficacy outcomes in patients with claudin 18.2-positive G/GEJ cancer (Biomarker Set)

| | 2.2 mg/kg | 2.6 mg/kg | 3.0 mg/kg | Total |
|---|---|---|---|---|
| | (n = 32) | (n = 45) | (n = 16) | (n = 93*) |
| Median (IQR) treatment duration, | 4.1 (1.4-4.9) | 2.1 (0.8-6.1) | 3.8 (1.9-8.1) | 3.0 (1.4-6.1) |
| months | | | | |
| Median (IQR) follow-up, months | 5.5 (4.2-9.0) | 6.0 (3.8-8.1) | 7.7 (5.2-9.7) | 6.0 (4.2-8.3) |
| Progression-free survival | | | | |
| Events | 20 (63%) | 33 (73%) | 7 (44%) | 60 (65%) |
| Median (95% CI), months | 4.8 (3.6-6.0) | 3.3 (2.2-6.1) | 14.5 (3.0-NR) | 4.8 (3.4-6.1) |
| Overall survival | | | | |
| Events | 8 (25%) | 19 (42%) | 7 (44%) | 34 (37%) |
| Median (95% CI), months | NR (6.5-NR) | 8.5 (6.2-NR) | NR (5.2-NR) | NR (7.5-NR) |

Data are presented as n (%) or median (interquartile range, IQR) unless stated otherwise.
*Includes 3 patients with G/GEJ cancer from the dose-escalation phase. G/GEJ = gastric/gastroesophageal junction. CI = confidence interval. NR = not reached.

TABLE 8
Best overall response evaluation of CMG901 in G/GEJ cancer cohort (Efficacy Set)

| | 2.2 mg/kg | 2.6 mg/kg | 3.0 mg/kg | Total |
|---|---|---|---|---|
| | (n = 43) | (n = 47) | (n = 19) | (n = 109*) |
| BOR | | | | |
| CR | 0 | 0 | 2 (11%) | 2 (2%) |
| PR | 19 (44%) | 15 (32%) | 6 (32%) | 40 (37%) |
| SD | 5 (12%) | 18 (38%) | 7 (37%) | 30 (28%) |
| PD | 14 (33%) | 13 (28%) | 4 (21%) | 31 (28%) |
| NE | 5 (12%) | 1 (2%) | 0 | 6 (6%) |
| ORR | 19 (44%; 29.1-60.1) | 15 (32%; 19.1-47.1) | 8 (42%; 20.3-66.5) | 42 (39%; 29.4-48.3)^{#} |
| Confirmed ORR | 13 (30%; 17.2-46.1) | 11 (23%; 12.3-38.0) | 6 (32%; 12.6-56.6) | 30 (28%; 19.4-36.9) |
| DCR | 24 (56%; 39.9-70.9) | 33 (70%; 55.1-82.7) | 15 (79%; 54.4-93.9) | 72 (66%; 56.4-74.9) |
| Confirmed DCR^{δ} | 24 (56%; 39.9-70.9) | 33(70%; 55.1-82.7) | 14 (74%; 48.8-90.9) | 71 (65%; 55.4-74.0) |
| Time to response | | | | |
| Number | 19 | 15 | 8 | 42 |
| Median (95% CI), | 2.0 (2.0-3.4) | 2.1 (2.3-NR) | 2.0 (2.0-NR) | 2.0 (2.2-NR) |
| months | | | | |
| Duration of response | | | | |
| Number | 19 | 15 | 8 | 42 |
| Median (95% CI), | 5.3 (2.6-7.2) | 4.3 (2.9-NR) | 12.6 (1.5-NR) | 5.6 (4.2-8.1) |
| months | | | | |

Data are presented as n (%) unless stated otherwise. Best responses of each patient assessed according to RECIST 1.1 by the investigator.
*Includes 6 patients with G/GEJ cancer from the dose-escalation phase.
^{#}Includes two patients who had the potential to achieve a confirmed PR.
^{δ}Calculated as the proportion of patients showing confirmed CR, PR, or SD for a minimum of 5 weeks from the first dosing date. G/GEJ = gastric/gastroesophageal junction; BOR = best overall response; CR = complete response; PR = partial response; SD = stable disease; PD = progressive disease; NE = not evaluable; ORR (CR + PR) = objective response rate; CI = confidence interval; DCR (CR + PR + SD) = disease control rate; NR = not reached.

TABLE 9
Efficacy outcomes in G/GEJ cancer cohort (Full Analysis Set)

| | 2.2 mg/kg | 2.6 mg/kg | 3.0 mg/kg | Total |
|---|---|---|---|---|
| | (n = 44) | (n = 50) | (n = 19) | (n = 113*) |
| Median (IQR) treatment | 2.9 (0.8-4.3) | 2.2 (0.8-6.2) | 3.3 (1.6-8.0) | 2.7 (0.9-5.7) |
| duration, months | | | | |
| Median (IQR) follow-up, | 5.4 (4.1-8.9) | 6.1 (3.9-8.3) | 7.4 (5.0-10.0) | 5.7 (4.2-8.5) |
| months | | | | |
| Progression-free survival | | | | |
| Events | 31 (70%) | 35 (70%) | 10 (53%) | 76 (67%) |
| Median (95% CI), months | 3.7 (2.1-4.8) | 3.5 (2.9-6.1) | 5.0 (3.0-NR) | 3.7 (3.3-4.9) |
| Overall survival | | | | |
| Events | 17 (39%) | 21 (42%) | 9 (47%) | 47 (42%) |
| Median (95% CI), months | 9.8 (5.5-NR) | 9.1 (6.2-NR) | NR (5.0-NR) | 9.8 (6.8-NR) |

Data are presented as n (%) or median (interquartile range, IQR) unless stated otherwise.
*Includes 6 patients with G/GEJ cancer from the dose-escalation phase. G/GEJ = gastric/gastroesophageal junction; CI = confidence interval; NR = not reached.

TABLE 10
Confirmed Responses in PDAC cohort

| | | Patients |
|---|---|---|
| | | (n = 38) |
| Dose Level (mg/kg) | 2.2 | 22 |
| | 2.6 | 11 |
| | 3.0 | 5 |
| Confirmed BOR | CR + PR | 4 |
| | SD | 14 |
| | PD | 15 |
| | NE | 5 |

TABLE 11
ORR in PDAC patients with ≥50%
CLDN18.2-positive tumour cells

| | 2.2 mg/kg | | 2.6 mg/kg | | 3.0 mg/kg | | Total | |
|---|---|---|---|---|---|---|---|---|
| | (n = 26) | | (n = 11) | | (n = 6) | | (n = 40) | |
| ORR | 2 | (8.7%) | 0 | | 2 | (33.3%) | 4 | (10.0%) |
| 95% CI, % | (1.1, 28.0) | | (0.0, 28.5) | | (4.3, 77.7) | | (2.8, 23.7) | |
| DCR | 11 | (47.8%) | 4 | (36.4%) | 3 | (50.0%) | 18 | (45.0%) |
| 95% CI, % | (26.8, 69.4) | | (10.9, 69.2) | | (11.8, 88.2) | | (29.3, 61.5) | |
| 2L population | 8 | | 3 | | 2 | | 13 | |
| ORR | 1 | (12.5%) | 0 | | 1 | (50.0%) | 2 | (15.4%) |
| 95% CI, % | (0.3, 52.7) | | (0.0, 70.8) | | (1.3, 98.7) | | (1.9, 45.4) | |
| DCR | 5 | (62.5%) | 1 | (33.3%) | 1 | (50.0%) | 7 | (53.8%) |
| 95% CI, % | (24.5, 91.5) | | (0.8, 90.6) | | (1.3, 98.7) | | (25.1, 80.8) | |
| 3L+ population | 18 | | 10 | | 6 | | 34 | |
| ORR | 1 | (5.6%) | 0 | | 1 | (16.7%) | 2 | (5.9%) |
| 95% CI, % | (0.2, 31.9) | | (0.0, 36.9) | | (0.6, 80.6) | | (0.9, 24.3) | |
| DCR | 6 | (33%) | 3 | (30%) | 2 | (33.3%) | 11 | (32.3%) |

| | | | | |
|---|---|---|---|---|
| 95% CI,% | (16.3, 67.7) | (8.5, 75.5) | (6.8, 93.2) | (22.4, 61.2) |

Data are presented as n (%) unless stated otherwise.

TABLE 12
Efficacy outcomes in PDAC patients with ≥50% CLDN18.2-positive tumour cells

| | 2.2 mg/kg | 2.6 mg/kg | 3.0 mg/kg | Total |
|---|---|---|---|---|
| | (n = 24) | (n = 12) | (n = 6) | (n = 42) |
| Progression-free survival | | | | |
| Events | 15 (62.5%) | 9 (75.0%) | 3 (50.0%) | 27 (64.3%) |
| Median (95% CI), months | 3.4 (0.7-4.0) | 2.3 (0.7-5.7) | 4.4 (0.7-NR) | 3.3 (1.6-3.5) |
| Overall Survival | | | | |
| Events | 6 (25%) | 6 (50%) | 2 (33.3%) | 14 (33.3%) |
| Median (95% CI), months | NR (5.1-NR) | 4.3 (2.8-NR) | NR(4.04, NR) | NR (4.30-NR) |
| 6 months OS (%, 95% CI) | 59.1 (17.6-85.3) | 46.7 (11.5-76.5) | 50 (5.8-84.5) | 52.6 (28.0-72.2) |
| 9 months OS (%, 95% CI) | 59.1 (17.6-85.3) | NR | NR | 52.6 (28.0-72.2) |

1. A method of treating cancer in a human subject, comprising administering to the subject an antibody-drug conjugate (ADC) comprising:
(i) an antibody or antigen-binding fragment thereof which specifically binds human claudin 18.2 and comprises a heavy chain variable region comprising VHCDR1, VHCDR2 and VHCDR3 and a light chain variable region comprising VLCDR1, VLCDR2 and VLCDR3, wherein:
VHCDR1 comprises the amino acid sequence set forth in SEQ ID NO: 1;
VHCDR2 comprises the amino acid sequence set forth in SEQ ID NO: 2;
VHCDR3 comprises the amino acid sequence set forth in SEQ ID NO: 3;
VLCDR1 comprises the amino acid sequence set forth in SEQ ID NO: 4;
VLCDR2 comprises the amino acid sequence set forth in SEQ ID NO: 5; and
VLCDR3 comprises the amino acid sequence set forth in SEQ ID NO: 6; and
(ii) monomethyl auristatin E (MMAE);
wherein the subject is administered at least one dose of the ADC of about 0.3 to about 3.4 mg/kg.

2. The method of claim 1, wherein;
(i) the antibody or antigen-binding fragment thereof is humanized;
(ii) the ADC comprises an antibody comprising a human IgG constant domain, preferably a human IgG1 constant domain; and/or
(iii) the MMAE is joined to the antibody or antigen-binding fragment thereof via a linker.

3. The method of claim 1, wherein the antibody or antigen-binding fragment thereof comprises:
(a) a heavy chain variable domain comprising the amino acid sequence set forth in SEQ ID NO: 7, or a variant thereof having at least 80, 85, 90 or 95% identity to SEQ ID NO: 7; and
(b) a light chain variable domain comprising the amino acid sequence set forth in SEQ ID NO: 8, or a variant thereof having at least 80, 85, 90 or 95% identity to SEQ ID NO: 8.

4. The method of claim 3, wherein the antibody or antigen-binding fragment thereof comprises:
(a) a heavy chain variable domain comprising the amino acid sequence set forth in SEQ ID NO: 7; and
(b) a light chain variable domain comprising the amino acid sequence set forth in SEQ ID NO: 8.

5. (canceled)

6. The method of claim 1, wherein the antibody comprises:
(a) a heavy chain comprising the amino acid sequence set forth in SEQ ID NO: 9, or a variant thereof having at least 80, 85, 90 or 95% identity to SEQ ID NO: 9; and
(b) a light chain comprising the amino acid sequence set forth in SEQ ID NO: 10, or a variant thereof having at least 80, 85, 90 or 95% identity to SEQ ID NO: 10.

7. The method of claim 6, wherein the antibody comprises:
(a) a heavy chain comprising the amino acid sequence set forth in SEQ ID NO: 9; and
(b) a light chain comprising the amino acid sequence set forth in SEQ ID NO: 10.

8. (canceled)

9. The method of claim 2, wherein the linker is 6-maleimidocaproyl-valine-citrulline-p-aminobenzyloxycarbonyl (MC-vc-PAB).

10. The method of claim 9, wherein the ADC comprises an antibody comprising:
(a) a heavy chain comprising the amino acid sequence set forth in SEQ ID NO: 9; and
(b) a light chain comprising the amino acid sequence set forth in SEQ ID NO: 10.

11. The method of claim 1, wherein the ADC has an average drug-to-antibody ratio (DAR) of 3.3 to 4.3.

12. The method of claim 11, wherein the ADC has an average DAR of 3.8.

13. The method of claim 1, wherein the subject is administered at least one dose of the ADC of about 0.3 mg/kg, 0.6 mg/kg, 1.2 mg/kg, 1.8 mg/kg, 2.2 mg/kg, 2.6 mg/kg, 3.0 mg/kg or 3.4 mg/kg.

14. The method of claim 1, wherein the subject is administered at least one dose of the ADC of about 1.8 to about 3.4 mg/kg.

15. The method of claim 14, wherein the subject is administered at least one dose of the ADC of about 1.8 mg/kg, 2.2 mg/kg, 2.6 mg/kg, 3 mg/kg or 3.4 mg/kg.

16. The method of claim 1, wherein the subject is administered a dose of the ADC about every 2 weeks, every 3 weeks or every 4 weeks.

17. The method of claim 16, wherein the subject is administered a dose of the ADC about every 3 weeks.

18. The method of claim 17, wherein the subject is administered a dose of the ADC of about 1.8 mg/kg, 2.2 mg/kg, 2.6 mg/kg, 3 mg/kg or 3.4 mg/kg about every 3 weeks.

19. The method of claim 16, wherein the subject is:
(a) administered the ADC for a duration of 1 to 12 months;
(b) administered the ADC in a total of at least 2 doses, at least 3 doses, at least 4 doses or at least 14 doses of the ADC;
(c) administered the ADC until confirmed disease progression, unacceptable toxicity or death; and/or
(d) administered the ADC intravenously.

20-23. (canceled)

24. The method of claim 1, wherein the cancer is a solid cancer.

25. The method of claim 1, wherein the cancer expresses claudin 18.2.

26. The method of claim 24, wherein the cancer is gastric cancer, gastroesophageal junction cancer or pancreatic cancer.

27. (canceled)

28. The method of claim 1, wherein the cancer is a locally unresectable or metastatic cancer.

29. The method of claim 1, wherein the cancer does not express HER2.

30. The method of claim 1, wherein the subject has received at least 2 prior lines of treatment.

31. The method of claim 26, wherein the method:
(a) has an overall response rate (ORR) of at least 30%;
(b) has a disease control rate (DCR) of at least 60%;
(c) gives a median progression-free survival of at least 3 months; and/or
(d) is associated with a median overall survival of at least 9 months.

32-40. (canceled)


SEQUENCE LISTING (13 sequences)

SEQ ID NO: 1 - protein, 11 aa, synthetic construct
GGSISSNYAWN

SEQ ID NO: 2 - protein, 16 aa, synthetic construct
YIYYSGNTNYNPSLKS

SEQ ID NO: 3 - protein, 9 aa, synthetic construct
SYYGNSFIY

SEQ ID NO: 4 - protein, 17 aa, synthetic construct
KSSQSLLNSGNQKNYLT

SEQ ID NO: 5 - protein, 7 aa, synthetic construct
WASTRES

SEQ ID NO: 6 - protein, 9 aa, synthetic construct
QNAYSFPWT

SEQ ID NO: 7 - protein, 118 aa, synthetic construct
QVQLQESGPGLVKPSETLSLTCTVSGGSISSNYAWNWIRQPPGKGLEWIGYIYYSGNTNYNPSLKSRVTISRDTSKNQFSLKLSSVTAADTAVYYCATSYYGNSFIYWGQGTLVTVSS

SEQ ID NO: 8 - protein, 113 aa, synthetic construct
DIVMTQSPDSLAVSLGERATINCKSSQSLLNSGNQKNYLTWYQQKPGQPPKLLIYWASTRESGVPDRFSGSGSGTDFTLTISSLQAEDVAVYYCQNAYSFPWTFGQGTKVEIK

SEQ ID NO: 9 - protein, 448 aa, synthetic construct
QVQLQESGPGLVKPSETLSLTCTVSGGSISSNYAWNWIRQPPGKGLEWIGYIYYSGNTNYNPSLKSRVTISRDTSKNQFSLKLSSVTAADTAVYYCATSYYGNSFIYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 10 - protein, 220 aa, synthetic construct
DIVMTQSPDSLAVSLGERATINCKSSQSLLNSGNQKNYLTWYQQKPGQPPKLLIYWASTRESGVPDRFSGSGSGTDFTLTISSLQAEDVAVYYCQNAYSFPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 261 aa, Homo sapiens
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 12 - protein, 261 aa, Homo sapiens
MSTTTCQVVAFLLSILGLAGCIAATGMDMWSTQDLYDNPVTSVFQYEGLWRSCVRQSSGFTECRPYFTILGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 13 - protein, 5 aa, synthetic construct
GGGGS